(19) United States
(12) Patent Application Publication
(10) Pub. No.: US 2023/0018584 A1
(43) Pub. Date: Jan. 19, 2023

(54) DEVICE FOR MEASURING VITAL PARAMETERS WITH ADVANTAGEOUS RADIATION GUIDANCE

(71) Applicant: PULSION MEDICAL SYSTEMS SE, Feldkirchen (DE)

(72) Inventors: Aaron Weber, Markt Schwaben (DE); André Hein, Esslingen am Neckar (DE); Thomas Thalmeier, Dorfen (DE)

(73) Assignee: PULSION MEDICAL SYSTEMS SE, Feldkirchen (DE)

(21) Appl. No.: 17/780,951

(22) PCT Filed: Nov. 30, 2020
(86) PCT No.: PCT/EP2020/083973

§ 371 Date: Aug. 10, 2022

(30) Foreign Application Priority Data
Dec. 1, 2019 (DE) 10 2019 008 332.6

(51) Int. Cl. A61B 5/024; A61B 5/021; A61B 5/00

(52) U.S. Cl. CPC A61B 5/02416; A61B 5/02108; A61B 5/7278; A61B 5/6826; A61B 2562/0238

(57) ABSTRACT

The present invention relates to a positioning and exposure device (1) for the defined arrangement on at least one body part (2, 3) of a living being (4) and for exposing the body part (2, 3) to radiation for determining at least one vital parameter of the living being (4). The positioning and exposure device (1) here comprises at least: a guiding and support structure (6) for delimiting an examination area (8), wherein the body part (2) can be positioned during exposure in the examination area (8), wherein the guiding and support structure (6) in a section (10) bounding the examination area (8) forms at least one radiation input area (12), wherein radiation can be introduced through the radiation input area (12) into the examination area (8) and wherein the guiding and support structure (6) forms a radiation exit area (16) in a further portion (14) delimiting the examination area (8), wherein at least part of the radiation which can be introduced through the radiation input area (12) into the examination area (8) can be guided out of the examination area (8) through the radiation exit area (16), and wherein a first elongate optical guide (18) is arranged in the path (20) of the radiation at least before entry into the examination area (8), wherein the first optical guide (18) is curved at least in sections for at least one deflection of the path (20) of the radiation which can be introduced into the first optical guide (18).

### TECHNICAL FIELD

The present invention relates to a measuring device and a supply device of such a measuring device, in particular for detecting vital parameters of living beings. The detection of vital parameters can preferably be carried out non-invasively by the measuring device. The supply device is particularly preferably a device which supplies radiation and/or pressure for determining blood pressure, in particular a supply device for supplying radiation and/or pressure for continuously determining the intra-arterial blood pressure on at least one finger of a hand. The measuring device is preferably a blood pressure measuring device, in particular a measuring device for continuously determining the intra-arterial blood pressure on at least one finger of a hand.

### PRIOR ART

The (in particular arterial) blood pressure of a patient is one of the most important measured variables in medical technology, and known, in particular also non-invasive, measuring technology associated with this is extremely diverse. This applies above all to measuring technology for continuously monitoring blood pressure over a prolonged period of time, for example in intensive care medicine, but also in emergency medicine and during surgical interventions.

For reasons of good accessibility, the blood pressure measuring device is often attached to a limb of a patient, for example an applanation tonometric sensor of the radial artery on the forearm or a finger sensor operated in a photoplethysmographic manner according to the so-called “Vascular Unloading Technique” according to Penaz. Such pressure measuring devices are known, for example, from U.S. Pat. Nos. 4,406,289, 4,524,777, 4,726,382, WO 2010/050798 A1, WO 2000/059369 A1, WO 2011/045138 A1, WO 2011/051819 A1, WO 2011/051822 A1, WO 2012/032413 A1, and WO 2017/143366 A1.

In the Vascular Unloading Technique, near-infrared light is radiated into a finger and the pulsatile (pulse-shaped) blood flow (actually the changing blood volume) in the finger is determined from the non-absorbed portion captured by means of a photodetector. For this process, also known as photoplethysmography (PPG), the (near-infrared) light is usually generated using one or more light-emitting diodes (LEDs), which work with one or more wavelengths, and is detected using one or more light-sensitive receiver diodes (photodiodes). Other types of photoreceivers besides diodes are also suitable in principle.

A control system now keeps constant the plethysmographically registered flow (or the detected blood volume) and thus the resulting photoplethysmographic signal (volume signal v(t)) by applying counterpressure in a cuff (cuff pressure) pc(t) on the finger. This counterpressure pc(t) is usually controlled by a high-speed valve or valve system in conjunction with a pump. The relevant control of the valve or valve system is carried out by a control unit, which is preferably implemented using a microcomputer. The main input signals are the PPG signal v(t) and the cuff pressure pc(t). The pressure pc(t) required to keep the PPG signal v(t) constant now corresponds to the intra-arterial blood pressure pa(t).

For this it is necessary that the cuff pressure pc(t) can be changed at least as fast as the intra-arterial blood pressure pa(t) changes, so that the real-time condition is satisfied. The upper limit frequency of pa(t), and thus the highest rate of pressure change, is greater than at least 20 Hz, which is quite a challenge for a pressure control system. From this it follows that the pressure control using a valve or valve system is advantageously disposed in the immediate vicinity of the cuff. If the air lines are too long, there is a risk that this limit frequency condition will be lost due to the low-pass effect of the lines.

A mechanical valve known from U.S. Pat. No. 4,406,289 regulates the counterpressure in the finger cuff with the desired accuracy when it is supplied with a linearly operating pump. The valve is housed in a housing on the distal forearm and thus supplies the finger cuff with the pressure pc(t) via a short hose.

U.S. Pat. No. 4,524,777 describes a pressure generation system for the vascular unloading technique, wherein a constant cuff pressure Pc is also generated with a linear pump and is superimposed with pressure fluctuations Δpc(t) from a “shaker” or a “driving actuator” connected in parallel.

U.S. Pat. No. 4,726,382 discloses a finger cuff for the vascular unloading technique which has hose connections for supplying the cuff pressure pc(t). The length of the air hoses extends to the pressure generation system, which in turn is attached to the distal forearm.

WO 2000/059369 A1 also describes a pressure generation system for the vascular unloading technique. The valve system here comprises a separate inlet valve and a separate outlet valve. While a relatively linear proportional pump must be used in patent specifications U.S. Pat. Nos. 4,406,289 and 4,524,777, this system allows the use of simple, inexpensive pumps, since disruptive harmonics can be eliminated by the arrangement of the valves. Furthermore, the energy consumption of the simple pump can be significantly reduced by the valve principle.

A system for the vascular unloading technique is known from WO 2004/086963 A1 in which the blood pressure can be continuously determined in one finger, while the measurement quality is checked in the adjacent finger (“watch dog” function). After a period of time, the system automatically changes the “measuring finger” to the “monitoring finger”.

WO 2005/037097 A1 describes a control system for the vascular unloading technique having a number of intertwined control loops.

WO 2010/050798 A1 discloses a pressure generation system attached to the distal forearm (“front end”) and having only one valve, to which a finger cuff for the vascular unloading technique can be attached.

In a pressure generation system for the vascular unloading technique described in WO 2011/045138 A1—similar to that known from WO 2000/059369—the energy consumption of the pump is reduced and harmonics can be eliminated.

WO 2011/051819 A1 discloses an implementation of the vascular unloading technique that has been improved by means of digital electronics in order to increase stability and for further miniaturization.

WO 2011/051822 A1 describes a method for the vascular unloading technique in which the measured signals v(t) and pc(t) are processed to increase long-term stability and to determine other hemodynamic parameters. In particular, a method for eliminating effects originating from vasomotor changes in the finger arteries and a method for determining cardiac output (CO) are disclosed.

WO 2012/032413 A1 describes novel finger sensors that have a disposable part for single use. In this case, the cuff that comes into contact with the finger is accommodated in the disposable part for reasons of hygiene, whereas the associated pressure generation and pressure control system is accommodated in a reusable part. Accordingly, a separable pneumatic connection is to be provided here between the disposable part and the reusable part.

As a rule, the pressure generation and pressure control system in the prior art is attached to the distal forearm, proximal to the wrist, which has significant disadvantages: This location is often used for intravenous lines and the intra-arterial access at the distal end of the radial artery should also be free for emergencies. Such accesses can be blocked by the pressure generating and pressure control system and its attachment. In addition, the system can slip or tilt during operation. This can adversely affect how the sensors are seated. The seating of the sensors would also improve if the finger to be measured or the corresponding hand is in a certain rest position.

To overcome this problem, WO 2017/143366 A1 proposes a measuring system for the continuously determining the intra-arterial blood pressure on at least one finger of a hand, having at least one finger sensor, having a plethysmographic system, having at least one light source, preferably LED, having one or several wavelengths and at least one light sensor and at least one inflatable cuff, and having a pressure generation system with at least one valve controlled in real time using the plethysmographic system for generating a pressure in the cuff that is essentially equal to the intra-arterial blood pressure in the finger, wherein the measuring system has a housing with a surface that acts as a resting surface for the at least one finger and the adjacent regions of the palm. The hand rests on a resting surface under which are disposed essential components that were attached to the forearm in conventional systems.

Similar to WO 2012/032413 A1 mentioned above, the cuff is accommodated in a disposable part that can be separated from the housing (and thus from the resting surface). Correspondingly, a separable pneumatic connection between the disposable part and the reusable part is to be provided here, as well.

In the known systems, the light-emitting diodes and photodiodes for emitting and detecting the near-infrared measurement radiation, possibly embedded in transparent silicone, are arranged directly on the finger. When the light-emitting diodes and photodiodes are arranged in a reusable part, there is the problem that the exposed light-emitting elements have to be cleaned and disinfected before they can be reused. The need for an easy-to-clean design limits the degrees of freedom in the design. In addition, the need to accommodate the light-emitting and photodiodes in the immediate vicinity of the finger represents a limitation in the geometric design of the device. If the light-emitting diodes and photodiodes are arranged in a single-use article, on the other hand, there is the problem that electrical connections must be provided between the single-use article and the reusable base unit, and that the costs for manufacturing the single-use article increase. The heat input from electrical components that come into contact with the skin is also perceived as negative.

### DESCRIPTION OF THE INVENTION

In view of the limitations of conventional systems, it is the object of the present invention to improve measuring devices of the type mentioned at the outset with respect to aspects of its manufacture and/or application.

According to one aspect of the present invention, this object is achieved with a device according to claim 1.

This device relates to a positioning and exposure device for the defined arrangement on at least one body part of a living being and preferably for exposing the body part to radiation for determining at least one vital parameter of the living being. The positioning and exposure device preferably has at least: A guiding and support structure for delimiting an examination area, wherein the body part can be positioned during exposure in the examination area. The guiding and support structure in a section delimiting the examination area preferably forms at least one radiation entry area. Radiation can preferably be introduced through the radiation entry area into the examination area. The guiding and support structure preferably forms a radiation exit area in a further section delimiting the examination area. Preferably, at least part of the radiation which can be introduced through the radiation entry area into the examination area can be guided out of the examination area through the radiation exit area. A first elongate optical guide is preferably arranged in the beam path of the radiation at least upstream of entry into the examination area. The first optical guide is particularly preferably curved, at least in sections, for at least one deflection of the beam path of the radiation which can be introduced into the first optical guide. Additionally or alternatively, a second elongate optical guide is arranged in the beam path of the radiation at least downstream of the radiation exit area. The second optical guide is preferably curved, at least in sections, for at least one deflection of the beam path of the radiation which can be introduced into the second optical guide.

Alternatively, the optical guide or optical guides, or a plurality of optical guides, can have deflection devices which cause the beam path to deflect without the specific optical guide having to be curved.

In this case, curved preferably describes a shape that deviates from a straight shape and can also be understood as arc-like or arc-shaped or bent. Alternatively or additionally, the presence of a deflection surface or a deflection portion for deflecting the radiation introduced into the specific optical guide can be described as curved.

Additionally or alternatively, the present invention can relate to a positioning and exposure device for defined arrangement on at least one body part of a living being and for exposing the body part to radiation and/or pressure for determining at least one vital parameter of the living being. This positioning and exposure device preferably has at least:

A guiding and support structure for delimiting an examination area and preferably for holding at least one force application device, wherein the body part can be positioned in the examination area during exposure, in particular exposure to radiation and/or pressure. The force application device is preferably connected to the guiding and support structure, it being possible for the body part to be subjected to pressure by means of the force application device. The guiding and support structure in a section delimiting the examination area preferably forms at least one radiation entry area, wherein radiation can be introduced through the radiation entry area into the examination area. In a further section delimiting the examination area, the guiding and support structure preferably forms a radiation exit area, wherein at least part of the radiation which can be introduced through the radiation entry area into the examination area can be guided out of the examination area through the radiation exit area.

A first, preferably elongate optical guide is preferably arranged in the beam path of the radiation at least before it enters the examination area, wherein the first optical guide is curved at least in sections for at least one deflection of the beam path of the radiation which can be introduced into the first optical guide.

Additionally or alternatively, a second, preferably elongate optical guide is arranged in the path of the radiation at least downstream of the radiation exit area, wherein the second optical guide is curved at least in sections for at least one deflection of the path of the radiation which can be introduced into the second optical guide.

According to one preferred embodiment of the present invention, the first optical guide has a first entry surface, a first main body, and a first exit surface, wherein the first main body and the first exit surface are oriented such that a main outward radiation direction out of the first optical guide is defined. Additionally or alternatively, the second optical guide has a second entry surface, a second main body, and a second exit surface, wherein the second main body and the second entry surface are preferably oriented such that a main inward radiation direction into the second optical guide is defined. The main outward radiation direction and the main inward radiation direction are particularly preferably oriented to one another at an angle of between 65° and 115°, in particular at an angle of between 75° and 105°, or at an angle of between 85° and 95°.

These solutions are advantageous because positioning and exposure devices can be provided for accommodating body parts of different sizes (in terms of volume), in particular fingers. These positioning and exposure devices preferably all have at least the first and/or the second optical guide. The positioning and exposure devices can be coupled, in particular detachably coupled, to a supply device one after the other or alternately. The first optical guides of the various positioning and exposure devices preferably have a first radiation entry surface, which is always arranged in the same position relative to the supply device when it is coupled to the supply device. The first entry surface or radiation entry surface is preferably always arranged in the region of the radiation source. Additionally or alternatively, the second optical guides of the various positioning and exposure devices preferably have a second exit surface or radiation exit surface, which is always arranged in the same relative position to the supply device when coupled to the supply device. The second radiation exit surface is preferably always arranged in the region of the detection device. Thus, the position of the detection device and/or the position of the radiation source can be fixed in the supply device and yet body parts of different sizes, in particular fingers, can be exposed to radiation for vital parameter analysis by means of different positioning and exposure devices.

The above-mentioned object is additionally achieved using a positioning and exposure device for defined arrangement on at least one body part of a living being, in particular for exposing the body part to radiation and/or pressure, in particular for determining at least one vital parameter of the living being. The inventive positioning and exposure device preferably has at least: One guiding and support structure for delimiting an examination area and preferably for holding at least one force application device, wherein the body part can be positioned in the examination area during exposure, in particular exposure to radiation and/or pressure. The force application device is preferably connected to the guiding and support structure, wherein pressure can be applied to the body part by means of the force application device.

The guiding and support structure in a section delimiting the examination area preferably forms at least one radiation entry area, wherein radiation can be introduced through the radiation entry area into the examination area. Additionally or alternatively, the guiding and support structure forms a radiation exit area in a further section delimiting the examination area, wherein radiation that can be introduced through the radiation entry area into the examination area can be guided out of the examination area through the radiation exit area.

The first optical guide preferably has a first entry surface, a first main body and a first exit surface, wherein the first main body and the first exit surface are oriented such that a main outward radiation direction out of the first optical guide is defined. Additionally or alternatively, the second optical guide has a second entry surface, a second main body, and a second exit surface, wherein the second main body and the second entry surface are oriented such that a main inward radiation direction into the second optical guide is defined. The main outward radiation direction and the main inward radiation direction are particularly preferably oriented to one another at an angle of between 65° and 115°, in particular at an angle of between 75° and 105° or at an angle of between 85° and 95° or at an angle of between 90° and 96°.

This solution is advantageous because it was recognized that the ideal angle between the main inward radiation direction and the main outward radiation direction for determining vital parameters is between 65° and 115°. In this range of angles, the proportions of reflected, transmitted, and scattered light are advantageous for analysis by means of a detection device such as a photodiode.

According to one further preferred embodiment of the present invention, a first elongate optical guide is arranged in the beam path of the radiation at least upstream of the examination area, wherein the first optical guide is curved at least in sections for at least one deflection of the beam path of the radiation which can be introduced into the first optical guide. Additionally or alternatively, a second elongate optical guide is arranged in the beam path of the radiation at least downstream of the radiation exit area, wherein the second optical guide is curved at least in sections for at least one deflection of the beam path of the radiation which can be introduced into the second optical guide.

The guiding and support structure is preferably a plastic component, in particular an inelastic plastic component, in particular an injection molded component. The guiding and support structure preferably forms one, exactly one or at least one, in particular two, exactly two or more than two, preferably ring-shaped wall structures, wherein the wall structure(s) preferably delimit through-openings or through-holes for introducing the body part or parts, in particular one or more fingers. Each through-opening receives exactly one body part, in particular a finger. Furthermore, the force application device is arranged or formed on the inside of the through-opening delimited by the wall structure. At least one force application device is preferably provided for each through-opening.

Furthermore, the wall structure forms the radiation entry area and the radiation exit area, in particular in the form of windows or through-holes. The wall structure particularly preferably forms the radiation entry area and the radiation exit area in the case of a plurality of through-openings, in particular in the case of at least two through-openings or exactly two through-openings or all through-openings.

Alternatively, the guiding and support structure can be designed as a preferably flexible cuff.

According to one further preferred embodiment of the present invention, at least the first optical guide and/or the second optical guide is/are fiber-free. This embodiment is advantageous since the radiation runs through a preferably homogeneous body and therefore no refraction effects occur.

According to one further preferred embodiment of the present invention, due to the curvature of the first optical guide, a first portion of the beam path is at an angle of more than 5°, in particular more than 10° or more than 15°, in particular in a range of between 5° and 85° and preferably in a range between 20° and 75°, or in a range between 30° and 60°, relative to the second portion of the beam path. The first portion of the beam path extends in the course of the beam path preferably at least immediately upstream of a first deflection region and wherein the second portion of the beam path in the course of the beam path preferably extends at least immediately downstream of the first deflection region. The deflection region is preferably designed as a component of the first optical guide. in particular as a surface or coating. This embodiment is advantageous because the radiation introduced or coupled into the optical guide can be deflected in a defined manner and can therefore be supplied to the examination area in a defined manner. According to the present invention, the terms “curvature” and “bend” are to be used synonymously.

According to one further preferred embodiment of the present invention, due to the curvature of the second optical guide, a third portion of the beam path is at an angle of greater than 5°, in particular greater than 10° or greater than 15°, in particular in a range between 5° and 85° and preferably in a range between 20° and 75° or in a range between 30° and 60°, relative to a fourth portion of the beam path. The third portion of the beam path preferably extends in the course of the beam path at least immediately upstream of a second deflection region and wherein the fourth portion of the beam path preferably extends in the course of the beam path at least immediately downstream of the second deflection region. The second deflection region is particularly preferably embodied as a component of the second optical guide. in particular as a surface or coating. This embodiment is advantageous because the distance from the center of the first exit surface of the first optical guide to the center of the second entry surface of the second optical guide (analogously for the further or second examination area) can be greater than the distance from the center of the first entry surface of the first optical guide to the center of the second exit surface of the second optical guide. The distance between the center of the first exit surface of the first optical guide and the center of the second entry surface of the second optical guide can preferably be at least 1.2 times or exactly 1.2 times or up to 1.2 times or at least 1.5 times or exactly 1.5 times or up to 1.5 times or at least 1.8 times or exactly 1.8 times or up to 1.8 times or at least 2 times or exactly 2 times or up to 2 times or at least 2.5 times or exactly 2.5 times or up to 2.5 times greater than the distance between the center of the first entry surface of the first optical guide and the center of the second exit surface of the second optical guide.

The first optical guide and/or the second optical guide and/or the third optical guide and/or the fourth optical guide is/are preferably embodied in one piece. Particularly preferably, the first optical guide and/or the second optical guide and/or the third optical guide and/or the fourth optical guide is/are a body, in particular made of glass, ceramic, and/or plastic, and is/are at least partially transparent to light radiation, in particular at least or precisely in the wavelength range from 500 nm to 2600 nm. Furthermore, the body can have one or more elements acting reflectively and/or diffractively on the radiation. These reflective and/or diffractive elements can be completely or partially enclosed or form a partial or complete coating.

Another preferred radiation range can be, e.g., between 1000 nm and 2600 nm, in particular between 1000 nm and 1600 nm or between 1600 nm and 2600 nm. This area can be used, for example, to determine lactate and/or glucose.

Another additional or alternative radiation range can be, e.g., between 600 nm and 1200 nm, in particular 750 nm and 1100 nm, in particular between 800 nm and 925 nm, in particular between 805 nm and 905 nm. This range can be used, e.g., for plethysmographic methods. Such methods are, e.g., ICG [=indocyanine green fluorescent dye] for liver function diagnostics, in which radiation in the range between 805 nm and 905 nm is preferably used. Additionally or alternatively, the vascular unloading technique (NIR, near infrared range), in this method, radiation is used in the range between 800 nm and 940 nm, in particular between 880 nm and 900 nm, in particular at 890 nm, essentially 890 nm or exactly 890 nm. Additionally or alternatively, multi-wavelength diagnostics, in which radiation in the range between 500 nm and 1100 nm, in particular in the range between 600 nm and 1000 nm, in particular in the range between 700 nm and 900 nm, is preferably used. Additionally or alternatively, in pulse oximetry (red visible light and NIR near infrared range) in which preferably radiation is used in the range between 640 nm and 680 nm, in particular in the range between 650 nm and 660 nm, in particular at 660 nm or essentially 660 nm or exactly 660 nm, and/or in the range between 890 nm and 970 nm, in particular in the range between 910 nm and 950 nm, in particular at 940 nm or essentially 940 nm or exactly 940 nm.

The radiation source can thus preferably emit radiation in the wavelength ranges of 500-1100 nm. This radiation can be used, e.g., to determine vital parameters with respect to pulse oximetry and/or pulse plethysmography. Pulse plethysmography is preferably carried out in the 800-940 nm range, in particular in the 880-900 nm range or essentially or exactly at 890 nm.

Pulse oximetry is preferably carried out in one or more ranges. The radiation source is thus preferably designed to emit radiation in the 640-680 nm range, in particular in the range between 650 and 660 nm, or in the range between 655 and 665 nm, or essentially or precisely at 660 nm. Additionally or alternatively, the radiation source is designed to emit radiation in the 910-950 nm range, in particular in the 920-945 nm or 935-945 nm range, or essentially or exactly at 940 nm. The radiation source can thus preferably emit radiation in a number of ranges, the radiation source thus preferably has a number of radiation elements, in particular LEDs or OLEDs, through which radiation can be emitted in one, two, three, several or all of the aforementioned ranges.

For the purposes of the present invention, vital parameters are, e.g., the activity of organs, in particular blood pressure, and/or the saturation and/or concentration of substances in the body, in particular in body fluids, e.g. blood, urine, saliva, or semen, and/or in body tissue, such as, e.g., muscle or fat tissue and/or organ tissue and/or bone, scalp, nail, hair, and/or tooth composition, in particular, e.g., oxygen saturation, lactate concentration, and/or glucose concentration.

According to one further preferred embodiment of the present invention, the first optical guide and/or a further first optical guide has portions having sectional surface areas of different sizes in its direction of longitudinal extension. The sectional surface area in the region of the first entry surface is preferably greater than in a central portion formed between the first entry surface and the first exit surface. Particularly preferably, the first optical guide or the further first optical guide forms a tapering portion that extends starting from the first entry surface in the direction of the first exit surface, wherein the sectional surface area preferably decreases, in particular continuously, starting from the first entry surface, in the direction of the first exit surface. This embodiment is advantageous because, on the one hand, a large entry surface can be provided for introducing or coupling in the radiation and one or more tapering portions can then be connected thereto in a way that saves material and weight.

According to one further preferred embodiment of the present invention, in its direction of longitudinal extension the second optical guide and/or the further second optical guide has portions with sectional surface areas of different sizes. The sectional surface area in the region of the second entry surface is preferably greater than in a central portion formed between the second entry surface and the second exit surface. Particularly preferably, the second optical guide and/or the further second optical guide forms a tapering portion that extends starting from the second entry surface in the direction of the second exit surface, wherein the sectional surface area preferably decreases, in particular continuously, starting from the second entry surface, in the direction of the second exit surface. This embodiment is advantageous because, on the one hand, a large second entry surface can be provided for introducing or coupling in the radiation and one or more tapering portions can then be connected thereto in a way that saves material and weight.

The first optical guide preferably has a constant sectional portion in its direction of longitudinal extension, wherein the constant sectional portion is preferably embodied between the tapering portion and the first exit surface.

The beam path of an ideally radiated and ideally forwarded light wave, in particular a directed light wave, is particularly preferably understood here as the beam path.

According to one further preferred embodiment of the present invention, at least a first surface portion of the first optical guide and/or at least a second surface portion of the second optical guide is matted, structured, and/or coated.

In this context, structured can mean, e.g., increased friction. Alternatively or additionally, structured can mean the presence of grooves and/or linear elevations, in particular rounded off at the ends, and/or nubs or depressions. The structuring of the first optical guide can particularly preferably form a Fresnel structure or Fresnel lens.

The “second” surface portion of the second optical guide generally describes a surface portion. To avoid confusion, the first optical guide essentially only has “first” surface portions (entry surface, etc.) and the second optical guide essentially only has “second” surface portions (entry surface, etc.). Thus, the “second surface” of the second optical guide is merely a name for the surface, but in terms of quantity it can also be the “first” surface of the second optical guide. This also applies analogously to other devices and elements.

According to one further preferred embodiment of the present invention, the first optical guide and the second optical guide differ in at least one and preferably in two, exactly two or more than two, or three or exactly three or more than three or up to three of the following parameters: Shape, in particular curvature, volume, mass, length and/or material. This embodiment is advantageous because it enables radiation guidance that is adapted to the supply device and the respective body sizes with a small and therefore resource-saving use of material.

According to one further preferred embodiment of the present invention, the first optical guide has a converging lens, wherein the converging lens preferably embodies the first entry surface. This embodiment is advantageous because it means that a large proportion of the radiation emitted by the radiation source can be conducted into the examination area.

According to one further preferred embodiment of the present invention, two optical guide pairs are provided. The first optical guide pair is preferably formed by the first optical guide and the second optical guide and the second optical guide pair is preferably formed by a further first optical guide and the second optical guide or a further second optical guide. The first optical guide pair and the second optical guide pair are preferably arranged such that different parts of the body, in particular fingers, are exposed to radiation alternately. Alternatively, the optical guides of the second optical guide pair can also be referred to as the first optical guide and the second optical guide.

This embodiment is advantageous because two body parts can be arranged in two examination areas at the same time. Radiation and/or pressure can preferably be applied to both body parts at the same time or at different times in order to analyze the vital parameters. This means that the radiation can preferably be coupled into both body parts at the same time or with a time offset. Additionally or alternatively, each body part can be subjected to a compressive force at the same time or with a time offset, or a compressive force for compressing the body parts is generated, in particular with force application devices. If the second optical guide pair is formed by a further first optical guide and the second optical guide, then the second optical guide is preferably arranged between the first optical guide and the further first optical guide. The first optical guide and the second optical guide and/or the first optical guide and the further first optical guide and/or all optical guides are preferably arranged in the same plane or run at least in the same plane in sections or extend at least in the same plane in sections. The axial center of the individual optical guides preferably extends in the same plane or in planes which are spaced apart from one another by less than 20 mm, in particular less than 10 mm or less than 5 mm or less than 1 mm.

According to one further preferred embodiment, the radiation entry area and the radiation exit area and the force application device lie in the same plane, at least in sections. This embodiment is advantageous because the vessels inside the body part can be compressed by the force application device in the region in which the radiation also penetrates into that body part. The radiation that is transmitted, reflected, and/or scattered through the compressed region of the body part is preferably guided to the detection device in this way.

If two optical guide pairs are provided, a radiation entry area and a radiation exit area are preferably provided for each optical guide pair or formed in the guiding and support structure.

If two optical guide pairs are provided, preferably at least one force application device is provided for each optical guide pair.

Particularly preferably, the first optical guide and the second optical guide and/or the first optical guide and the further first optical guide and/or all optical guides and each radiation entry area and each radiation exit area and the force application device are arranged in the same plane or run in the same plane, at least in sections, or extend in the same plane, at least in sections.

According to one further preferred embodiment of the present invention, a holder device is provided. The holder device is preferably embodied for the defined arrangement of at least two optical guide pairs. The holder device preferably has a functional material, in particular pigment, wherein the functional material absorbs radiation in the wavelength range of at least 500 nm to 1100 nm, in particular in the range between 600 nm and 960 nm or in the range between 600 nm and 800 nm, in particular in the range between 630 nm and 700 nm, or in the range between 800 nm and 1000 nm, in particular in the range between 820 nm and 960 nm, in particular in the range between 840 nm and 940 nm, in particular in the range between 870 nm and 910 nm, in particular in the range between 885 nm and 895 nm, in particular essentially or exactly 890 nm. Alternatively, the functional material can absorb radiation in a number of wavelength ranges, in particular wavelength ranges that are spaced apart from one another. The holder device preferably has a light blocker batch which preferably contains inorganic and/or organic pigments, in particular carbon black and/or titanium dioxide, in particular 2-15% by volume, in particular 3-8% by volume, or 2-15% by weight, especially 3-8% by weight.

According to one further preferred embodiment of the present invention, the holder device encloses the optical guides, at least in sections, preferably on exactly or at least two sides, at least in sections, preferably largely, in particular more than 50% (based on the surface area of the specific optical guide) or more than 75% or more than 90% or completely.

According to one further preferred embodiment of the present invention, the second optical guide and the further second optical guide are spaced closer to one another than the second optical guide is spaced from the further first optical guide or than the first optical guide is spaced from the further second optical guide.

According to one further preferred embodiment of the present invention, the optical guides are arranged in the holder device such that the axial center of the individual optical guides extend in the same plane or in planes that are spaced apart from one another by less than 20 mm, in particular less than 10 mm or less than 5 mm or less than 1 mm.

According to one further preferred embodiment of the present invention, the holder device has a radiation barrier, in particular a wall, between the second light device and the further second holder device. The radiation barrier preferably prevents the further, second optical guide from being acted upon by leakage radiation which escapes from the first optical guide. In addition or alternatively, the radiation barrier prevents the second optical guide from being acted upon by leakage radiation which escapes from the other first optical guide.

Definition:

Leakage radiation refers here to radiation that does not exit from an optical guide via the first exit surface or the second exit surface.

### DESCRIPTION

Functional material: Pigment: Which one and in what concentration (%/Vol or %/kg)

The holder device encloses the optical guides, in particular partially or completely, only in the region between the entry surface and the exit surface.

Furthermore, the present invention can relate to a positioning and exposure device for defined arrangement on at least one body part of a living being and for exposing the body part to radiation and pressure, in particular for determining at least one vital parameter of the living being. The positioning and exposure device preferably has at least:

A guiding and support structure for delimiting an examination area and preferably for holding at least one force application device, wherein the body part can be positioned in the examination area during exposure, in particular exposure to radiation and/or pressure. The force application device is preferably connected to the guiding and support structure, wherein the body part can preferably be exposed to pressure by means of the force application device.

The guiding and support structure in a section delimiting the examination area preferably forms at least one radiation entry area, wherein radiation can be introduced through the radiation entry area into the examination area.

Additionally or alternatively, the guiding and support structure forms a radiation exit area in a further section delimiting the examination area. Radiation that can be introduced or guided into the examination area through the radiation entry area can preferably be guided out of the examination area through the radiation exit area.

A holder device is particularly preferably provided or is a component of the positioning and exposure device. The holder device is preferably embodied for the defined arrangement of at least two optical guide pairs. The holder device preferably has a functional material, in particular pigment, wherein the functional material absorbs radiation in the wavelength range of at least 500 nm to 1040 nm, in particular in the range between 600 nm and 960 nm or in the range between 600 nm and 800 nm, in particular in the range between 630 nm and 700 nm, or in the range between 800 nm and 1000 nm, in particular in the range between 820 nm and 960 nm, in particular in the range between 840 nm and 940 nm, in particular in the range between 870 nm and 910 nm, in particular in the range between 885 nm and 895 nm, in particular essentially or exactly 890 nm. Alternatively, the functional material can absorb radiation in a number of wavelength ranges, in particular wavelength ranges that are spaced apart from one another.

The holder device preferably encloses the optical guide(s), at least in sections, on at least two sides. The holder device preferably encloses the optical guide or guides, at least in sections, preferably largely, in particular more than 50% (based on the surface area of the specific optical guide, or more than 75% or more than 90% or completely (based on the surface area of the specific optical guide).

Additionally or alternatively, the second optical guide and the further second optical guide are spaced closer to one another than the second optical guide is spaced from the further first optical guide or the first optical guide is spaced from the further second optical guide.

The optical guides are preferably arranged in the holder device such that the axial center of the individual optical guides extend in the same plane or in planes, which are spaced apart by less than 20 mm, in particular less than 10 mm or less than 5 mm or less than 1 mm.

At least one optical guide is preferred and a plurality of or all optical guides are preferably arranged or embodied in a fixed or detachable manner and/or in one piece or in multiple pieces in the holder device or on the holder device.

The holder device preferably forms a radiation barrier, in particular a wall, between the second light device and the further second optical guide, wherein the radiation barrier prevents the further second optical guide from being acted upon by leakage radiation which escapes from the first optical guide or prevents the second optical guide from being acted upon by leakage radiation which escapes from the further first optical guide.

Furthermore, the present invention can relate to a supply device for supplying radiation and preferably a functional fluid, in particular for determining vital parameters, in particular for coupling to a positioning and exposure device described herein, in particular a positioning and exposure device according to claim 1. The supply device preferably has at least:

A first radiation source and a first detection device, in particular a radiation detection device, and a holder device.

The holder device is preferably designed for the defined arrangement of at least two optical guide pairs,

The holder device is preferably designed for the defined arrangement of at least two optical guide pairs. The holder device preferably has a functional material, in particular pigment, wherein the functional material absorbs radiation in the wavelength range of at least 500 nm to 1040 nm, in particular in the range between 600 nm and 960 nm or in the range between 600 nm and 800 nm, in particular in the range between 630 nm and 700 nm, or in the range between 800 nm and 1000 nm, in particular in the range between 820 nm and 960 nm, in particular in the range between 840 nm and 940 nm, in particular in the range between 870 nm and 910 nm, in particular in the range between 885 nm and 895 nm, in particular essentially or exactly 890 nm. Alternatively, the functional material can absorb radiation in a number of wavelength ranges, in particular wavelength ranges that are spaced apart from one another.

The holder device preferably encloses the optical guide or guides, at least in sections, on at least two sides. The holder device preferably encloses the optical guide or guides, at least in sections, preferably largely, in particular more than 50% (based on the surface area of the specific optical guide), or more than 75% or more than 90% or completely (based on the surface area of the specific optical guide).

Additionally or alternatively, the second optical guide and the further second optical guide are spaced closer to one another than the second optical guide is spaced from the further first optical guide or the first optical guide is spaced from the further second optical guide.

The optical guides are preferably arranged in the holder device such that the axial center of the individual optical guides extend in the same plane or in planes, which are spaced apart by less than 20 mm, in particular less than 10 mm or less than 5 mm or less than 1 mm.

At least one optical guide is preferred and a plurality of or all optical guides are preferably arranged or embodied in a fixed or detachable manner and/or in one piece or in multiple pieces in the holder device or on the holder device.

The holder device preferably forms a radiation barrier, in particular a wall, between the second light device and the further second optical guide, wherein the radiation barrier prevents the further second optical guide from being acted upon by leakage radiation which escapes from the first optical guide or prevents the second optical guide from being acted upon by leakage radiation which escapes from the further first optical guide.

In addition, the supply device can supply or have or comprise a functional fluid supply device. The functional fluid supply device is preferably provided for supplying a fluid to a force application device, in particular the positioning and/or supply device. The supply device preferably forms a housing or has a housing, wherein the functional fluid supply device is preferably arranged in the interior of the housing. The radiation source and/or the detection device, in particular the first detection device and/or the second detection device, are additionally or alternatively preferably arranged in the housing of the supply device.

Furthermore, the present invention can relate to a measuring device for determining at least one vital parameter of a living being, in particular for continuously determining the intra-arterial blood pressure on at least one finger of a hand. The measuring device preferably has at least:

A supply device for supplying at least one radiation source for supplying radiation and particularly preferably for supplying a functional fluid supply device, in particular a functional fluid pump, and for supplying at least one detection device and one positioning and exposure device for the defined arrangement on at least one body part of a living being and for exposing the body part to the radiation and preferably pressure, wherein the positioning and exposure device has a guiding and support structure for delimiting an examination area and wherein the guiding and support structure preferably holds at least one force application device.

The supply device and the positioning and exposure device can preferably be physically coupled to and/or decoupled from one another without the use of tools and/or non-destructively.

The measuring device preferably has a holder device and at least one optical guide pair, the optical guide pair having a first optical guide and a second optical guide. The first optical guide preferably has a first entry surface, a first main body adjoining the first entry surface, and a first exit surface adjoining the first main body. Additionally or alternatively, the second optical guide preferably has a second entry surface, a second main body adjoining the second entry surface, and a second exit surface adjoining the second main body.

The holder device is preferably provided for the in particular defined arrangement of the at least one optical guide pair and preferably at least or exactly two optical guide pairs.

The holder device preferably positions the first entry surface adjacent to the radiation source and/or the first exit surface in front of the examination area or adjacent to the examination area. Additionally or alternatively, the holder device positions the second entry surface in front of the examination area or adjacent to the examination area and/or the second exit surface adjacent to the detection device.

The holder device is preferably embodied for the defined arrangement of at least two optical guide pairs. The holder device preferably has a functional material, in particular pigment, wherein the functional material absorbs radiation in the wavelength range of at least 500 nm to 1040 nm, in particular in the range between 600 nm and 960 nm or in the range between 600 nm and 800 nm, in particular in the range between 630 nm and 700 nm, or in the range between 800 nm and 1000 nm, in particular in the range between 820 nm and 960 nm, in particular in the range between 840 nm and 940 nm, in particular in the range between 870 nm and 910 nm, in particular in the range between 885 nm and 895 nm, in particular essentially or exactly 890 nm. Alternatively, the functional material can absorb radiation in a number of wavelength ranges, in particular wavelength ranges that are spaced apart from one another.

The holder device preferably encloses the optical guide(s), at least in sections, on at least two sides. The holder device preferably encloses the optical guide(s), at least in sections, preferably largely, in particular more than 50% (based on the surface area of the specific optical guide), or more than 75% or more than 90% or completely (based on the surface area of the specific optical guide).

Additionally or alternatively, the second optical guide and the further second optical guide are spaced closer to one another than the second optical guide is spaced from the further first optical guide or the first optical guide is spaced from the further second optical guide.

The optical guides are preferably arranged in the holder device such that the axial center of the individual optical guides extend in the same plane or in planes, which are spaced apart by less than 20 mm, in particular less than 10 mm or less than 5 mm or less than 1 mm.

At least one optical guide is preferred and a plurality of or all optical guides are preferably arranged or embodied in a fixed or detachable manner and/or in one piece or in multiple pieces in the holder device or on the holder device.

The holder device preferably forms a radiation barrier, in particular a wall, between the second light device and the further second optical guide, wherein the radiation barrier prevents the further second optical guide from being acted upon by leakage radiation which escapes from the first optical guide or prevents the second optical guide from being acted upon by leakage radiation which escapes from the further first optical guide.

According to one further preferred embodiment of the present invention, at least a first radiation source and a first detection device, in particular a radiation detection device, are arranged in a supply device.

The radiation source is preferably arranged upstream of the first entry surface in the beam path of the radiation that can be generated by the first radiation source, and the detection device is preferably arranged downstream of the second exit surface in the beam path of the radiation that can be generated by the first radiation source.

Within the scope of the present invention, the supply device can alternatively be referred to as a base part. Within the scope of the present invention, the positioning and exposure device, in particular the guiding and support structure, can alternatively be referred to as a cuff part.

According to one further preferred embodiment of the present invention, a coupling device is provided for detachably coupling the guiding and support structure and the supply device at least in a positive fit and/or at least in a non-positive fit. At least the first optical guide and/or the second optical guide and/or a further first optical guide and/or a further second optical guide are preferably arranged on the guiding and support structure in a decoupled state.

According to one further preferred embodiment of the present invention, a coupling device is provided for detachably coupling the guiding and support structure and the supply device at least in a positive fit and/or at least in a non-positive fit. At least the first optical guide and/or the second optical guide and/or a further first optical guide and/or a further second optical guide are preferably arranged on the supply device in a decoupled state.

It is preferably possible to be able to decouple and/or couple the guiding and support structure and the supply device without tools and/or non-destructively, in particular repeatedly.

The positioning and exposure device and/or the supply device preferably has a pressure control system for controlling a fluid pressure of the functional fluid. The pressure control system for controlling a fluid pressure is preferably embodied or arranged in or on the guiding and support structure and/or in or on the supply device. In terms of the present invention, a gas, in particular air, or a liquid, in particular water or deionized water, is the functional fluid.

The above object is also achieved using a supply device according to claim 14. This supply device preferably supplies radiation and preferably supplies a functional fluid, in particular for determining vital parameters, in particular for coupling to a positioning and exposure device described herein, in particular a positioning and exposure device according to claim 1. The supply device preferably has at least: a first radiation source and a first detection device, in particular a radiation detection device, a first optical guide, and/or a second optical guide. The first optical guide is preferably arranged such that the radiation from the radiation source can be introduced into the first optical guide through a first entry surface of the first optical guide. wherein the radiation can be guided out of the first optical guide through a first exit surface of the first optical guide. The second optical guide is preferably arranged such that radiation that has exited from the first optical guide through the first exit surface of the first optical guide can be introduced into the second optical guide through a second entry surface of the second optical guide. wherein the radiation introduced into the second optical guide through the second entry surface can be guided out of the second optical guide through a second exit surface and can be conducted to the detection device. The first optical guide and/or the second optical guide is/are preferably curved.

Additionally or alternatively, a first main body extends between the first entry surface and the first exit surface, wherein the first main body and the first exit surface are oriented such that a main outward radiation direction is defined, and wherein a second main body extends between the second entry surface and the second exit surface, wherein the second main body and the second exit surface are oriented such that a main inward radiation direction is defined, wherein the main outward radiation direction and the main inward radiation direction are oriented relative to one another at an angle between 65° and 115°, in particular at an angle between 75° and 105° or at an angle between 85° and 95°.

In addition, the supply device can supply or have or comprise a functional fluid supply device. The functional fluid supply device is preferably provided for supplying a fluid to a force application device, in particular the positioning and/or supply device. The supply device preferably forms a housing or has a housing, wherein the functional fluid supply device is preferably arranged in the interior of the housing. The radiation source and/or the detection device, in particular the first detection device and/or the second detection device, are additionally or alternatively preferably arranged in the housing of the supply device.

The functional fluid supply device is preferably embodied as a valve device which regulates, in particular enables, limits, or prevents, the forwarding or further flow of the functional fluid, which is preferably supplied via a supply line. The functional fluid is preferably supplied via the supply line at a pressure that is higher than the ambient pressure.

Furthermore, the present invention can relate to a measuring device for determining at least one vital parameter of a living being, in particular for continuously determining the intra-arterial blood pressure on at least one finger of a hand. This measuring device preferably has at least:

One supply device for supplying at least one radiation source for supplying radiation and preferably for supplying a functional fluid supply device, in particular a functional fluid pump, and for supplying at least one detection device, and a positioning and exposure device for the defined arrangement on at least one body part of a living being and for exposing the body part to the radiation and preferably, wherein the positioning and exposure device comprises a guiding and support structure for delimiting an examination area and preferably for holding at least one force application device.

The supply device and the positioning and exposure device can preferably be physically coupled to and/or decoupled from one another without the use of tools and/or non-destructively.

Furthermore, the measuring device preferably has a first optical guide and a second optical guide and particularly preferably has a further first optical guide and a further second optical guide.

The first optical guide is preferably arranged such that the radiation from the radiation source can be introduced into the first optical guide through a first entry surface of the first optical guide, wherein the radiation can be guided out of the first optical guide through a first exit surface of the first optical guide. Additionally or alternatively, the second optical guide is arranged such that radiation that has exited the first optical guide through the first exit surface of the first optical guide can be introduced into the second optical guide through a second entry surface of the second optical guide, wherein the radiation introduced into the second optical guide through the second entry surface can be guided out of the second optical guide through a second exit surface and can be conducted to the detection device. The first optical guide and/or the second optical guide is/are preferably curved. Additionally or alternatively, a first main body extends between the first entry surface and the first exit surface, wherein the first main body and the first exit surface are oriented such that a main outward radiation direction is defined, and wherein a second main body extends between the second entry surface and the second exit surface, wherein the second main body and the second exit surface are oriented such that a main inward radiation direction is defined, wherein the main outward radiation direction and the main inward radiation direction are oriented relative to one another at an angle between 65° and 115°, in particular at an angle between 75° and 105° or at an angle between 85° and 95°.

A coupling device is preferably provided for at least detachably coupling the positioning and exposure device, in particular the guiding and support structure, and the supply device in a positive fit and/or in at least a force fit, wherein at least the first optical guide is arranged on the positioning and exposure device in a decoupled state. Alternatively, at least the first optical guide is arranged on the supply device in a decoupled state. Alternatively, at least the first optical guide is arranged, in a decoupled and/or coupled state, on a holder device and/or is arranged physically separated from the supply device and the positioning and exposure device.

One or more of the aforementioned embodiments can additionally or alternatively have the features in the following.

According to one further preferred embodiment of the present invention, there is no electrical line connection between the positioning and exposure device and the supply device. However, the positioning and exposure device can have an electronic component for wireless identification of the positioning and exposure device, for example an RFID tag, so that it can be ensured by means of an associated query element, which is preferably arranged in the supply device, that only suitable positioning and exposure devices are used in operation. Likewise, a component for identifying the positioning and exposure device can advantageously prevent the reuse of a positioning and exposure device designed as a disposable component.

Doing without electrical contacts between the supply device and positioning and exposure device can increase both patient safety and functional reliability.

Alternatively, the positioning and exposure device can advantageously also have an electronic component for identifying the positioning and exposure device, and an interface for querying the electronic component can be the only electrical line connection between the supply device and the positioning and exposure device.

According to one preferred embodiment, the radiation source and the detection device, which is preferably embodied as an optical detection device, in particular as a photodetector, can be arranged on a common circuit board. Particularly advantageously, a driver switch for the radiation source and/or an amplifier circuit for the detection device, in particular the photodetector, can also be arranged on the circuit board. Due to the typically low currents in the μA range, short line lengths are advantageous, in particular between the detection device, in particular the photodiode (photodetector), and the amplifier circuit, which, in addition to cost-effective production and compact design, also speaks in favor of equipping a common circuit board with the corresponding electronic components. In this case, the detection device can have, e.g., one or more photodiodes. The detection device is preferably an optical detection device, in particular a photodetector. A detection device is preferably assigned to every second optical guide, i.e., optical guides through which radiation from the examination area is guided to the detection device. The number of second optical guides preferably correlates to the number of detection devices. Alternatively, however, it is also possible that two second optical guides is guided through the radiation from the examination area to the detection device, are assigned to a detection device, or the radiation guided with the specific optical guide is conducted to a or precisely one detection device.

Preferably exactly one or at least one lens, in particular up to two or up to three lenses, in particular one or more converging lens(es) and/or one or more diffusing lens(es) can be provided or embodied between radiation source and associated optical guide connection or optical guide and/or between the detection device, in particular the photodetector, and associated optical guide connection or optical guide, and additionally or alternatively a lens geometry can be integrated into the optical guide at the transition.

According to one further preferred embodiment of the present invention, one or more photocells and/or one or more photomultipliers and/or one or more CMOS sensors and/or one or more CCD sensors and/or one or more photodiodes and/or one or more phototransistors and/or one or more photoresistors can be used as the detection device, in particular as a photodetector.

The optical contact point for coupling light from the supply device into the positioning and exposure device and/or the optical contact point for decoupling near-infrared light from the positioning and exposure device into the supply device can advantageously additionally or alternatively be equipped with exactly one or at least one lens, in particular up to two or up to three lenses, in particular one or more converging lens(es) and/or one or more diffusing lens(es), additionally or alternatively a lens geometry can be integrated into the optical guide at the transition.

According to one advantageous refinement, an optical interface for coupling light from the supply device into the positioning and exposure device and/or an optical interface for decoupling light from the positioning and exposure device into the supply device is preferably provided with at least or precisely one cover glass, in particular with several cover glasses, in particular with up to two or one additional or up to three cover glasses. The cover glasses are preferably transparent at least for radiation in a wavelength range of at least 500 nm to 1040 nm, in particular in the range between 600 nm and 960 nm or in the range between 600 nm and 800 nm, in particular in the range between 630 nm and 700 nm, or in the range between 800 nm and 1000 nm, in particular in the range between 820 nm and 960 nm, in particular in the range between 840 nm and 940 nm, in particular in the range between 870 nm and 910 nm, in particular in the range between 885 nm and 895 nm, in particular essentially or exactly 890 nm. Furthermore, a cover glass or a plurality of cover glasses can have or be equipped with optical filters for filtering out defined radiation components, in particular radiation outside the wavelength range from 500 nm to 1040 nm, in particular outside the range from 600 nm to 960 nm or outside the range from 600 nm to 800 nm, in particular outside the range from 630 nm to 700 nm, or outside the range from 800 nm to 1000 nm, in particular outside the range from 820 nm to 960 nm, in particular outside the range from 840 nm to 940 nm, in particular outside the range from 870 nm to 910 nm, in particular outside the range from 885 nm to 895 nm, in particular substantially or exactly deviating from 890 nm. This is advantageous because one or a plurality of optical filters can prevent the detection device from being exposed to ambient light.

A cover glass or a plurality of cover glasses is/are preferably coated, in particular with an anti-reflection coating, which is preferably applied using the PVD method or by means of sputter deposition and which preferably has monocrystalline germanium or zinc selenide. The coating is preferably an IR AR coating, which is preferably adjusted to the range of 840-940 nm, in particular exactly or essentially 890 nm.

According to one further advantageous development, the optical emission surface or first or second radiation exit surface and/or the optical collector surface or first or second radiation entry surface is equipped with a Fresnel structure for directed coupling in or decoupling of the measurement radiation.

The above object is also achieved using a method according to claim 16. The method preferably relates to exposing living beings to radiation, in particular for determining at least one vital parameter, and preferably to pressure. The method preferably has at least the following steps:

Supplying a measuring device for determining at least one vital parameter of a living being, in particular for continuously determining the intra-arterial blood pressure on at least one finger of a hand, at least having one supply device for supplying at least one radiation source for supplying radiation and preferably for supplying a functional fluid supply device, in particular a functional fluid pump, and for supplying at least one detection device, and a positioning and exposure device for the defined arrangement on at least one body part of a living being and for exposing the body part to the radiation and preferably pressure, wherein the positioning and exposure device comprises a guiding and support structure for delimiting an examination area and preferably for holding at least one force application device. The supply device and the positioning and exposure device can preferably be physically coupled to and/or decoupled from one another without the use of tools and/or non-destructively.

Furthermore, the measuring device preferably has a first optical guide and a second optical guide and particularly preferably has a further first optical guide and/or a further second optical guide.

The first optical guide is preferably arranged such that the radiation from the radiation source can be introduced into the first optical guide through a first entry surface of the first optical guide, wherein the radiation can be guided out of the first optical guide through a first exit surface of the first optical guide. Additionally or alternatively, the second optical guide is preferably arranged such that at least some of the radiation that has exited the first optical guide through the first exit surface of the first optical guide can be introduced into the second optical guide through a second entry surface of the second optical guide.

Particularly preferably, the radiation introduced into the second optical guide through the second entry surface can be guided out of the second optical guide through a second exit surface and can be conducted to the detection device. The first optical guide and/or the second optical guide is/are preferably curved and the further first optical guide and/or the further second optical guide is/are particularly preferably curved.

Additionally or alternatively, a first main body extends between the first entry surface and the first exit surface, wherein the first main body and the first exit surface are oriented such that a main outward radiation direction is defined, and wherein a second main body extends between the second entry surface and the second exit surface, wherein the second main body and the second exit surface are oriented such that a main inward radiation direction is defined.

The main outward radiation direction and the main inward radiation direction are preferably oriented to one another at an angle of between 65° and 115°, in particular at an angle of between 75° and 105° or at an angle of between 85° and 95° . This applies analogously to the further first optical guide and the further second optical guide.

The method further includes the steps: emitting radiation using the radiation source, directing the radiation to an examination area, wherein the radiation is guided through the first optical guide, introducing radiation from the examination area into the second optical guide, and guiding the radiation introduced into the second optical guide to the detection device.

The above object is also achieved by a method according to claim 17. The method preferably relates to exposing living beings to radiation, in particular for determining at least one vital parameter, and preferably to pressure. The method preferably has at least the following steps:

Supplying a positioning and exposure device, wherein the positioning and exposure device has a guiding and support structure for delimiting an examination area and preferably for holding at least one force application device, wherein the body part is positioned in the examination area during the exposure, in particular exposure to radiation and/or application of pressure. The force application device is preferably connected to the guiding and support structure, wherein the guiding and support structure embodies at least one radiation entry area in a section delimiting the examination area.

The guiding and support structure preferably forms a radiation exit area in a further section delimiting the examination area.

A first elongate optical guide is preferably arranged in the beam path of the radiation at least upstream of the examination area, wherein the first optical guide is curved at least in sections for at least one deflection of the beam path of the radiation that can be introduced into the first optical guide. Additionally or alternatively, a second elongate optical guide is arranged in the beam path of the radiation at least downstream of the radiation exit area, wherein the second optical guide is curved at least in sections for at least one deflection of the beam path of the radiation which can be introduced into the second optical guide.

The method also preferably has the following steps:

Arranging in a defined manner at least one body part of a living being on or in the positioning and exposure device and exposing the body part to radiation, wherein radiation is introduced through the first radiation entry area and into the examination area out of the first optical guide for exposing the body part and wherein at least some of the radiation introduced into the examination area through the radiation entry area can be conducted through the radiation exit area and into the second optical guide, wherein the radiation introduced into the second optical guide is forwarded in a defined manner by the second optical guide.

The method may further include the step: Applying pressure to the body part, wherein the force application device applies pressure to the body part. This step is preferably carried out at the same time the body part is exposed to radiation.

The above object is also achieved by a method according to claim 18. The method preferably relates to exposing living beings to radiation, in particular for determining at least one vital parameter, and preferably to pressure. The method preferably has at least the following steps:

Supplying a positioning and exposure device, wherein the positioning and exposure device has a guiding support structure for delimiting an examination area and preferably for holding at least one force application device, wherein the body part is positioned in the examination area during the exposure, in particular the exposure to radiation and/or pressure. The force application device is preferably connected to the guiding and support structure. The guiding and support structure in a section delimiting the examination area preferably forms at least one radiation entry area. Additionally or alternatively, the guiding and support structure forms a radiation exit area in a further section delimiting the examination area. A first elongate optical guide is preferably arranged in the beam path of the radiation at least upstream of the examination area, wherein the first optical guide has a first entry surface, a first main body, and a first exit surface, wherein the first main body and the first exit surface are oriented such that a main outward radiation direction is defined. Additionally or alternatively, a second elongate optical guide is arranged in the beam path of the radiation at least downstream of the radiation exit area, wherein the second optical guide has a second entry surface, a second main body, and a second exit surface, wherein the second main body and the second entry surface are oriented such that a main inward radiation direction is defined.

The main outward radiation direction and the main inward radiation direction are particularly preferably oriented to one another at an angle of between 65° and 115°, in particular at an angle of between 75° and 105°, or at an angle of between 85° and 95°.

The method preferably also has at least the following steps:

Arranging in a defined manner at least one body part of a living being on the positioning and exposure device and exposing of the body part to radiation, wherein radiation is introduced through the first radiation entry area and guided out of the first optical guide into the examination area for exposing the body part and wherein at least some of the radiation introduced into the examination area through the radiation entry area can be conducted through the radiation exit area and into the second optical guide, wherein the radiation introduced into the second optical guide is forwarded in a defined manner by the second optical guide.

The method may further include the step: Applying pressure to the body part, wherein the force application device applies pressure to the body part. This step is preferably carried out at the same time the body part is exposed to radiation.

The above object is also achieved by a method according to claim 19. The method preferably relates to exposing living beings to radiation, in particular for determining at least one vital parameter, and preferably to pressure. The method preferably has at least the following steps:

Supplying a supply device for supplying radiation and a functional fluid, wherein the supply device has at least one radiation source and one detection device, in particular a radiation detection device, a first optical guide, and a second optical guide. The supply device can preferably also have a functional fluid supply device. The first optical guide is preferably arranged such that the radiation from the radiation source can be introduced into the first optical guide through a first entry surface of the first optical guide. The radiation can preferably be guided out of the first optical guide through a first exit surface of the first optical guide. The second optical guide is preferably arranged such that radiation that has exited from the first optical guide through the first exit surface of the first optical guide can be introduced into the second optical guide, preferably through a second entry surface of the second optical guide. Particularly preferably, the radiation introduced into the second optical guide through the second entry surface can be guided out of the second optical guide through a second exit surface and can be conducted to the detection device. The first optical guide and/or the second optical guide and/or a further first optical guide and/or a further second optical guide is/are preferably curved.

Additionally or alternatively, a first main body extends between the first entry surface and the first exit surface, wherein the first main body and the first exit surface are oriented such that a main outward radiation direction is defined. A second main body preferably extends between the second entry surface and the second exit surface, wherein the second main body and the second exit surface are preferably oriented such that a main inward radiation direction is defined. The main outward radiation direction and the main inward radiation direction are particularly preferably oriented to one another at an angle of between 65° and 115°, in particular at an angle of between 75° and 105°, or at an angle of between 85° and 95°.

The method also preferably has the following steps:

Emitting radiation through the radiation source and coupling the radiation into the first optical guide and detecting radiation by means of the detection device, wherein portions of the radiation emitted by the radiation source and coupled into the first optical guide are conducted to the detection device through the second optical guide.

It is also possible for the method to have the step of supplying the functional fluid through the functional fluid supply device.

Alternatively, it is possible for the inventive devices, i.e. the measuring device, the supply device, and/or the positioning and exposure device, and/or the inventive methods to be embodied or designed without a functional fluid supply device and/or without a force application device and/or without a functional fluid.

According to one preferred embodiment of the present invention, a force application device for applying pressure to the body part is preferably provided as a component of one of the positioning and exposure device(s) described herein. The positioning and exposure device can be, e.g., a component of a measuring device or can be a device that can be coupled to a supply device.

The force application device is preferably connected to the guiding and support structure of the positioning and exposure device and is particularly preferably held by the guiding and support structure.

Furthermore, the present invention according to claim 20 relates to a set. The set preferably has at least one first positioning and exposure device according to one of claims 1 to 9 and at least one second positioning and exposure device according to one of claims 1 to 9, wherein the shape and/or orientations of the optical guides of the first positioning and exposure device is different from the shape and/or orientations of the optical guides of the second positioning and exposure device. In addition, the set can have a supply device.

The supply device preferably has a coupling point to which the first positioning and exposure device can be detachably coupled and to which the second positioning and exposure device can be detachably coupled. Furthermore, a third positioning and exposure device according to one of claims 1 to 9 can be part of the set. The third positioning and exposure device can preferably also be detachably coupled to the coupling point of the supply device. Particularly preferably, the optical guides of the third positioning and exposure device have an orientation and/or shape that differs from the orientation and/or shape of the first and/or second positioning and exposure devices. A deviation in shape or orientation can be understood as any structural change or change in orientation that causes a different course of the beam path through the first and second optical guide of the respective positioning and exposure device. Thus, the beam path of the first positioning and exposure device differs from the beam path of the second positioning and exposure device and of the third positioning and exposure device.

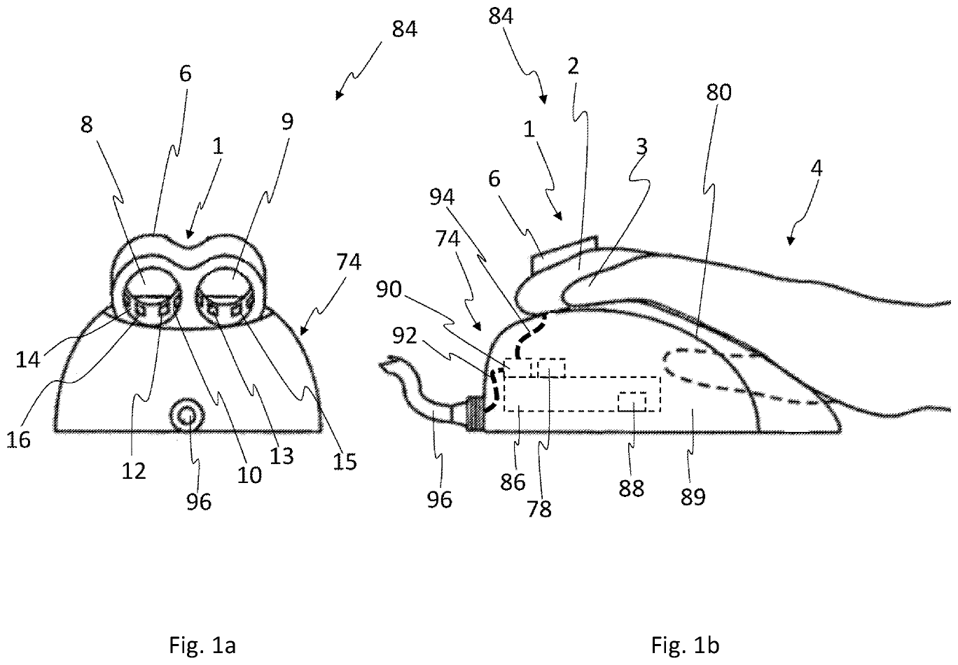

FIG. 1a shows a first schematic view of an example of an inventive measuring device 84. The measuring device 84 preferably has at least one and preferably more than one, in particular two or more than two or three or more than three, positioning and exposure devices 1 and a supply device 74.

The positioning and exposure device 1 preferably has at least one guiding support structure 6 for delimiting the examination area 8 or the examination areas 8 and 9. The guiding and support structure is preferably used to hold at least one force application device, in particular at least or exactly one force application device 82 per examination area 8, 9, wherein the body part 2, 3 can be positioned in the examination area 2, 3 during exposure, in particular exposure to radiation and/or pressure. The force application device 82 is preferably connected to the guiding and support structure 6, it being possible for the body part to be subjected to pressure by means of the force application device 82.

The force application device 82 is preferably embodied as a bladder, in particular as a plastic bladder. The bladder is preferably arranged on the guiding support structure and delimiting the examination area. The bladder is preferably arranged opposite the radiation entry area and/or the radiation exit area. The radiation entry area and/or the radiation exit area for each examination area (in the case of one examination area or several examination areas) is preferably arranged or embodied on one side of the examination area and the bladder or bladders are preferably arranged or embodied opposite thereto, in particular on the other side of the examination area. The bladder is preferably arranged vertically above the radiation entry area and/or the radiation exit area.

FIG. 1b

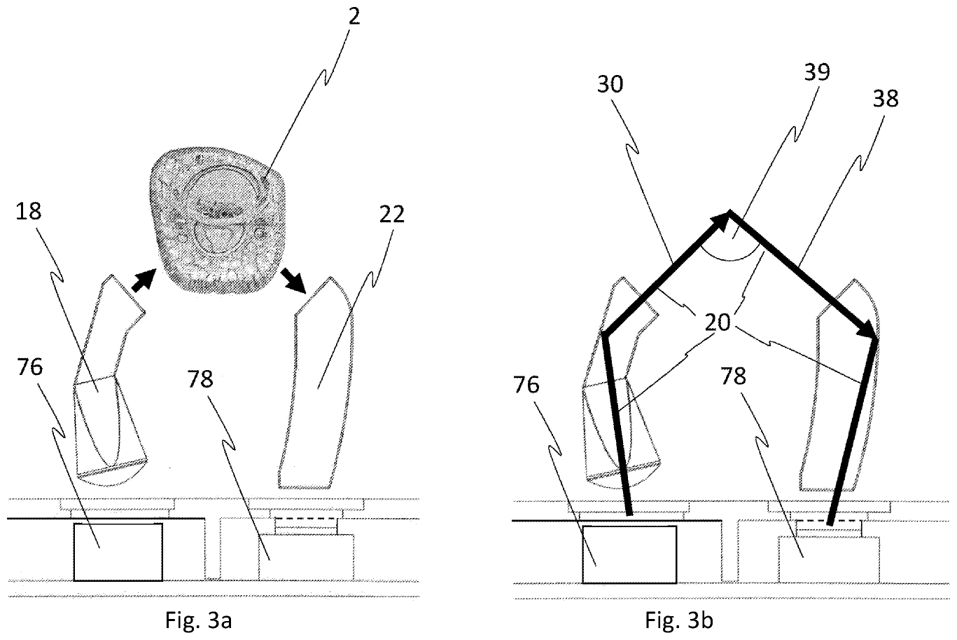

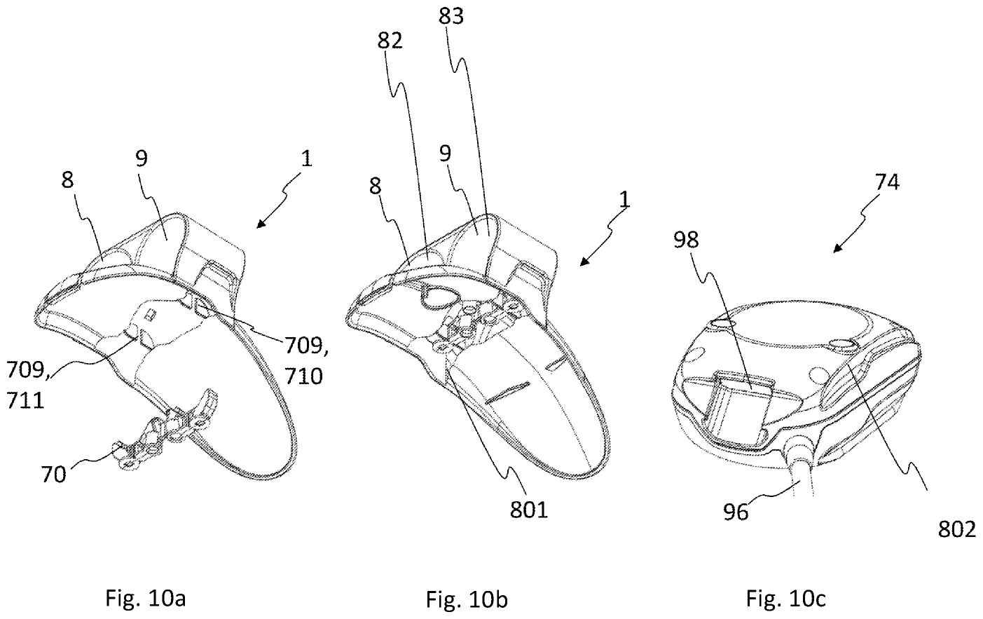

In addition to the exposure radiation, the supply device 74 preferably also provides at least one functional fluid, wherein one functional fluid supply device 90 is preferably arranged in the accommodation space 89, wherein the functional fluid device 90 is embodied to supply the functional fluid as a function of the detection radiation detected by detection device 78, wherein a control device 88 is preferably arranged in the accommodation space 89, wherein the control device 88 preferably controls the first radiation source 76 (see FIG. 3a) and/or the functional supply device 90, wherein the line connection 96 has a first supply line 92 for supplying current for operating the control device 88, the first radiation device 76 (see FIG. 3a), and the first detection device 78, and/or wherein the line connection 96 has a second or alternative supply line for supplying the functional fluid to the functional fluid supply device 90. It is additionally or alternatively possible for the functional fluid to be supplied via the first supply line. Furthermore, it is additionally or alternatively possible for a further line connection, in particular a detachable line connection, to be provided or embodied for supplying current for operating the control device 88, first radiation device 76 (see FIG. 3a), and/or first detection device 78 and/or for exchanging data. Additionally or alternatively, it is possible for an interface 98 (see FIG. 10c) to be provided or embodied for coupling a further line connection, in particular for supplying current for operating the control device 88 and/or the first radiation device 76 (see FIG. 3a) and/or the first detection device 78 and/or for exchanging data. The functional fluid supply device 90 is preferably embodied as a valve device which controls, in particular enables, limits, or prevents, the forwarding or further flow of the functional fluid which is preferably supplied via a supply line 92, 96. The functional fluid is preferably supplied via the supply line at a pressure that is higher than the ambient pressure.

For the purposes of the present invention, the term exposure radiation is preferably understood to mean the radiation which is supplied by a radiation source 76 (see FIG. 3a), in particular the first radiation source and/or a further radiation source, and/or which is conducted to a body part 2, 3 or emitted in the direction of an exposure point or an examination area 8, 9. In the context of the present invention, detection radiation 78 is understood to be the portion of the exposure radiation scattered or deflected by the body part 2, 3 or that passed through the body part and is conducted or supplied to the detection device 78 or reaches the detection device 78.

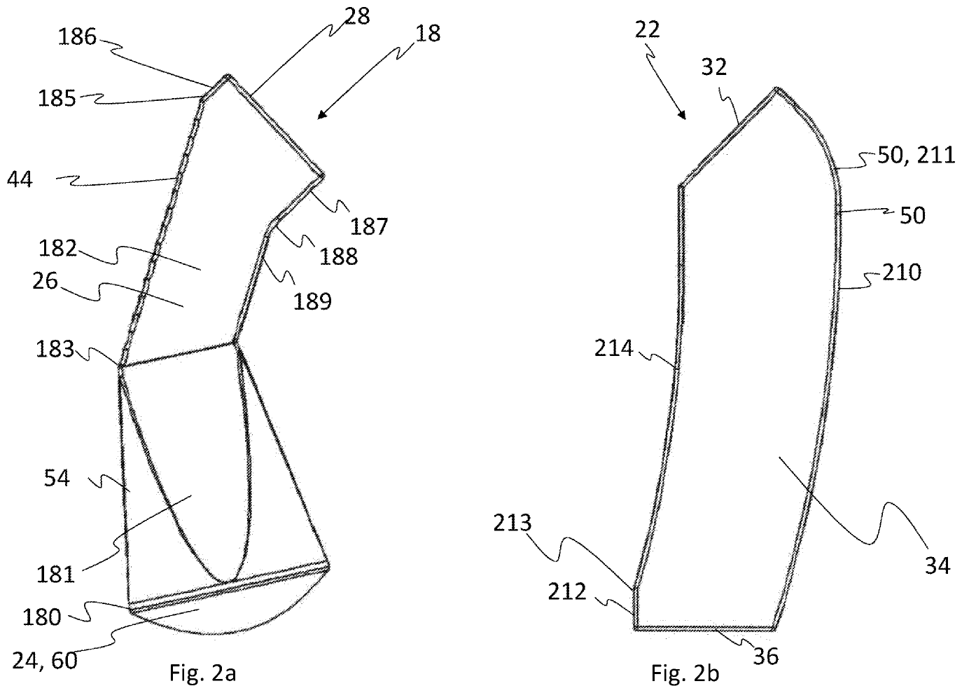

FIG. 2a shows an example of a first optical guide 18. The first optical guide 18 has an entry surface 24 and an exit surface 28 spaced apart from one another longitudinally. The entry surface 24 and the exit surface 28 are preferably oriented relative to one another at an angle of between 3° and 85°, in particular at an angle of between 10° and 80°, in particular at an angle of between 45° and 80°. A main body 26 extends between the entry surface 24 and the exit surface 28. In a sectional representation, in particular in a longitudinal sectional representation, the main body has a first, in particular external, contour which extends between the entry surface 24 and the exit surface 28. Furthermore, the main body 26 has a second, in particular interior, contour.

“Interior” is preferably to be understood as facing a further optical guide of the same optical guide pair and “external” is preferably to be understood as facing away from the further optical guide of the same optical guide pair. The same understanding can apply to optical guides of a further optical guide pair, in particular when a further optical guide pair is provided.

The entry surface 24 preferably has a curved shape. Particularly preferably, the entry surface 24 is part of or a component of a lenticular portion of main body 26. A preferably tapering portion 54 or an at least partially conical portion preferably adjoins the entry surface 24, in particular the lens-shaped portion, in the region of a transition 180. The tapering portion 54 or the conical portion can have one or a plurality of partially flattened or planar surfaces 181. Reference number 183 designates an end of the tapering portion 54 or of the conical portion and/or the beginning of a further portion 182, which is in particular tubular or cylindrical, at least in sections. The portion 182 preferably embodies a deflection region 44 for deflecting the radiation. The deflection region 44 can preferably be coated and/or structured. A further preferably cylindrical and/or tubular and/or conical region 186 preferably adjoins the portion 182 via a transition point 185. Region 186 is preferably shorter than the deflection portion 44. Alternatively, it is also possible for the deflection region 44 to extend, in particular in a straight line or in the form of a cylinder, to the exit area 28. The deflection portion 44 is preferably longer or shorter than the tapering portion 54. Alternatively, the tapering portion 54 and the deflection portion 44 can be of the same length. Furthermore, at least or exactly one further portion can be embodied between the deflection portion 44 and the tapering portion 54. Furthermore, the tapering portion 54 and/or the deflection portion 44 and/or the region 186 can be curved. Furthermore, the tapering portion 54 is oriented relative to the deflection portion at an angle of less than 85°, in particular less than 80° or less than 70° or less than 60° or less than 50° or less than 45°.

In the interior, starting from the exit region 28, the contour preferably has an end region 187, in particular a straight or curved region 187. The end region 187 is preferably adjacent to a transition point 188, via which the end region 187 is connected to a further region, preferably further straight region 189, in particular the ear-shaped or cylindrical portion. One, two or more than two, or up to two or three or exactly three or up to three or more than three or all of these portions and/or regions can be coated and/or structured.

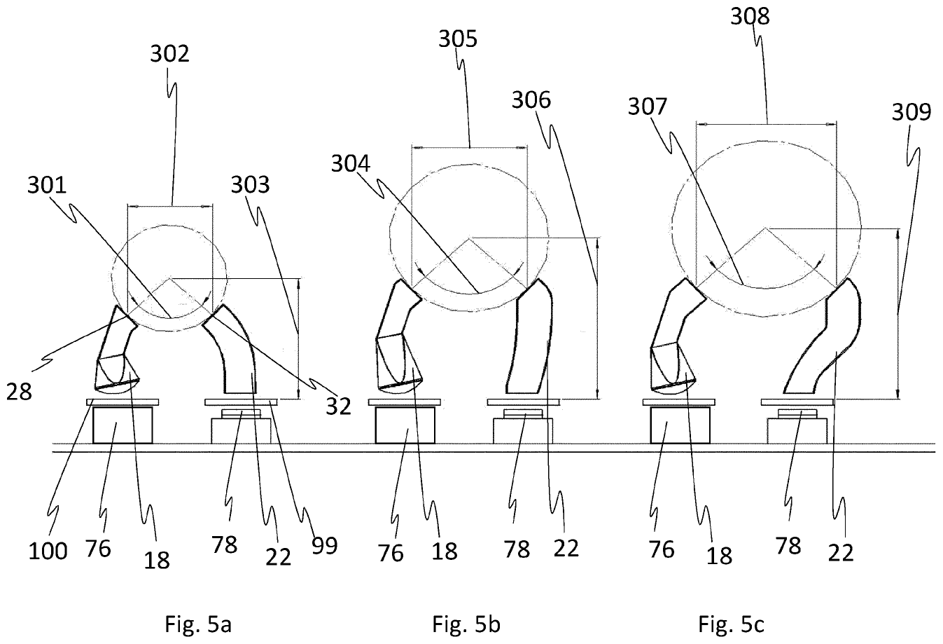

FIG. 2b shows an example of a second optical guide 22, in particular an optical guide as shown in FIG. 5b. This optical guide 22 has a second entry surface 32, a second main body 34 and a second exit surface 36. The entry surface 32 and the exit surface 36 are preferably spaced apart from one another in the longitudinal direction of the main body 34. The entry surface 32 and exit surface 36 are preferably oriented at an angle to one another, in particular in an angular range between 85° and 3°, in particular in an angular range between 80° and 45° or in an angular range between 75° and 50°.

This optical guide 22 preferably has an interior contour portion which, starting from the entry surface 32, preferably extends in a curved manner or in curved sections or in straight sections or in completely straight sections. This is then followed, preferably via a transition point 213, by a portion 212 which extends preferably straight or straight in sections or completely curved or in sections that are curved. Furthermore, it is alternatively possible for the portion 214 or only the portion 214 to connect the entry surface 32 to the exit surface 36. Alternatively, it is also possible for the portion 212 or only the portion 212 to connect the entry surface 32 to the exit surface 36. Furthermore, a further portion or a plurality of further portions can be embodied between the portion 212 and the portion 214. On the exterior, the second optical guide 22 preferably has a first preferably curved portion 211 and a second preferably curved portion 210 adjoining it, in particular continuously. The portion 211 preferably has a greater curvature than the portion 210. Alternatively, however, it is also possible for one or more regions to be provided. Additionally or alternatively, the portion 211 and/or the portion 210 can be formed in a straight line. Furthermore, the portion 210 preferably has a length which corresponds to a multiple of, in particular at least 1.5 times or at least 2 times or at least 3 times, the length of the portion 211.

FIG. 3a shows a schematic representation according to which the radiation is introduced into a body part 2 from the first optical guide 18. The body part 2 deflects portions of the radiation so that they penetrate into the second optical guide 22.

FIG. 3b shows an ideal or preferred beam path 20 starting from the radiation source 76 and ending at the detection device 78. This illustration also shows that an angle 39 is formed between the main outward radiation direction 30 and the main inward radiation direction 36. The second optical guide 22 (and, analogously, a further second optical guide 23) is preferably oriented such that a main inward radiation direction 36 is specified on the basis of the orientation and structural shape.

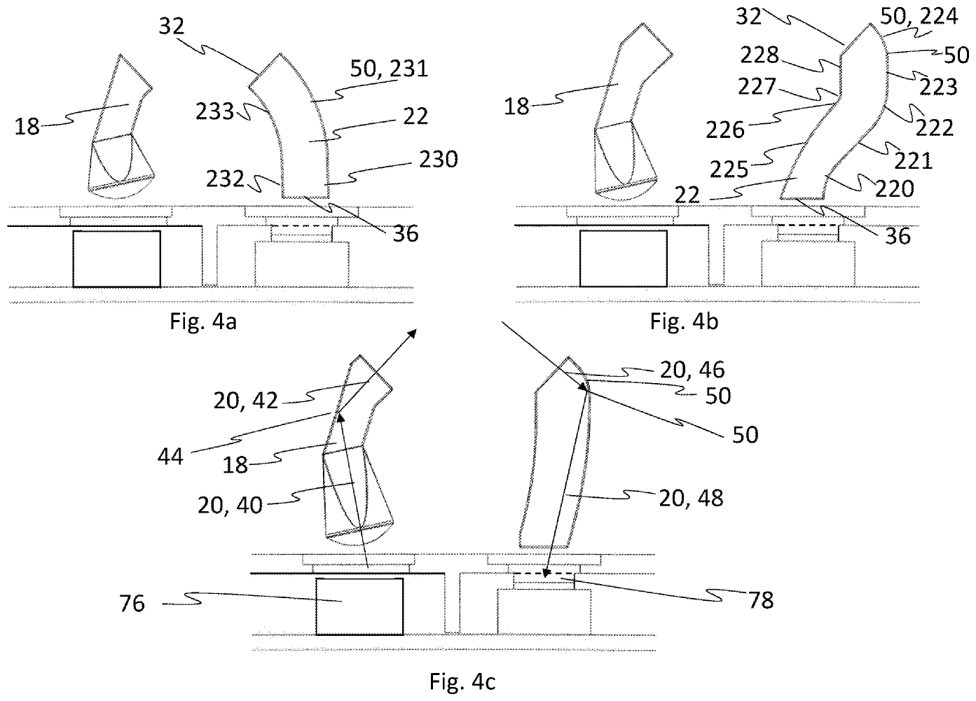

FIG. 4a shows an optical guide pair which can preferably correspond to the optical guide pair shown in FIG. 5a. In its interior, the second optical guide 22 preferably has a straight portion 232 and an adjoining curved portion 233, which preferably transition into one another continuously. The straight portion 232 ends on the one hand at the exit surface 36 and the curved portion 232 ends on the one hand at the entry surface 32. The contour of the optical guide 22 can preferably also only be curved on the interior. The curved portion 233 is preferably longer than the straight portion 232 by a multiple, in particular at least 1.5 times or at least 2 times or at least 3 times longer.

The exterior contour of the optical guide 22 preferably also has a curved portion 231 and a straight portion 230, which preferably transition into one another continuously. The straight portion 230 ends on the one hand at the exit surface 36 and the curved portion 231 ends on the one hand at the entry surface 32. The contour of the optical guide 22 can preferably also only be curved on the exterior. The curved portion 231 is preferably longer than the straight portion 230 by a multiple, in particular at least 1.5 times or at least 2 times or at least 3 times longer.

The thickness of the optical guide 22 is preferably continuous.

FIG. 4b shows an optical guide pair which can preferably correspond to the optical guide pair shown in FIG. 5c. The optical guide 22 extends in its longitudinal direction from an entry surface 32 to an exit surface 36. A straight portion 228 preferably adjoins the entry surface on the interior. The portion 228 transitions into a bend 227 toward the exit surface 36. The bend 227 preferably adjoins a turning point 226, which preferably adjoins a further bend 225. The further bend 225 is preferably curved in a different direction than the first bend 227. The bend 225 preferably extends in length by a multiple, in particular at least 1.5 times or at least 2 times or at least 3 times, greater than the bend 227 and/or the straight portion 228. It is alternatively possible for the interior contour to have only one bend 227 or 225 or other bends in addition to the bends 227 and 225. Furthermore, the portion 228 can also be embodied bent and/or the portion 225 can be embodied straight.

On the exterior, a bend 224 preferably adjoins the entry surface 32, which is preferably adjoined by a straight portion 223. A bent portion 222 preferably adjoins the straight portion 223, wherein the portion 222 is preferably bent in the same direction as the portion 224. Adjoining the portion 222 is preferably a straight portion 221, which preferably has or forms a turning point. A further bent portion 220 then preferably adjoins the straight portion 221, wherein this further bent portion 220 is bent in a different direction than the portion 224 and/or the portion 222. Alternatively, it is possible for the entire exterior contour to have or form a bend or two opposing bends that preferably transition directly into one another. The thickness of the optical guide 22 preferably changes in the longitudinal direction of the optical guide 22. A first length portion of the optical guide 22 extending from the entry surface in the direction of the exit surface and over 50% of the length preferably has more material forming the optical guide 22 in terms of mass and/or volume than a second length portion extending from the exit surface in the direction of the entry surface over 50% of the length of the optical guide. Alternatively, a first length portion of the optical guide 22 extending from the entry surface in the direction of the exit surface and over 50% of the length has less material forming the optical guide 22 in terms of mass and/or volume than a second length portion extending from the exit surface in the direction of the entry surface over 50% of the length of the optical guide.

FIG. 4c shows an optical guide pair which can preferably correspond to the optical guide pair shown in FIG. 5b. Due to the curvature or bending of the first optical guide 18, a first portion 40 of the beam path 20 is preferably oriented relative to a second portion 42 of the beam path 20 at an angle of greater than 5°, in particular greater than 10° or greater than 15°, in particular in a range between 5° and 85° and preferably in a range between 20° and 75° or in a range between 30° and 60°. The first portion 40 of the beam path 20 preferably extends in the course of the beam path 20 at least immediately upstream of a first deflection region 44, and the second portion 42 of the beam path 20 in the course of the beam path 20 extends at least immediately downstream of the first deflection region 44.

FIGS. 5a to 5c shows three differently designed optical guide pairs. The optical guide pair in FIG. 5a is preferably part of a first positioning and exposure device 1. The optical guide pair in FIG. 5b is preferably part of a second positioning and exposure device 1. The optical guide pair in FIG. 5c is preferably part of a third positioning and exposure device 402.

It can be seen that the angle 301 is smaller than the angle 304 or the angle 307, wherein the angle 304 is smaller than the angle 307. It can also be seen that the distance 302 is less than the distance 305 or the distance 308, wherein the distance 305 is smaller than the distance 308. Furthermore, it can be seen that the height 303 is less than the height 306 and the height 309, wherein the height 306 is less than the height 309. The angles 301, 304, and 307 each indicate an angle between the main outward radiation direction 30 and the main inward radiation direction 36. The distances 302, 305, and 308 each indicate a distance from the center of the first exit surface 28 and the second entry surface 32. The heights 303, 306, and 309 preferably indicate the distance of a point of intersection between the main outward radiation direction 30 and the main inward radiation direction 36 to a surface of a cover glass 99.

In the sense of a set, several positioning devices 1, 400, 402, in particular differently configured, can preferably be provided. In this case, e.g., the first positioning and exposure device 1 has at least one optical guide pair that differs in shape and/or orientation from an optical guide pair of the second positioning and exposure device 400. The second positioning and exposure device 400 preferably has at least one optical guide pair that differs in shape and/or orientation from an optical fiber pair of the third positioning and exposure device 402. The first positioning and exposure device 1 preferably has at least one optical guide pair that differs in shape and/or orientation from an optical guide pair of the third positioning and exposure device 402.

It is possible here for the guiding and support structure 6 of the individual positioning and exposure devices 1, 400, 402 to be configured identically, but for each to have different holder devices 70. In this case, the first positioning and exposure device 1 can have a holder device 70 which has one or more optical guide pairs which are oriented and/or embodied in a first configuration, in particular in the configuration shown in FIG. 5a. The second positioning and exposure device 400 can have a holder device 70 which has one or more optical guide pairs which are oriented and/or embodied in a second configuration, in particular in the configuration shown in FIG. 5b. The third positioning and exposure device 402 can have a holder device 70 which has one or more optical guide pairs which are oriented and/or embodied in a third configuration, in particular in the configuration shown in FIG. 5c. The first, second, and third configurations preferably differ.

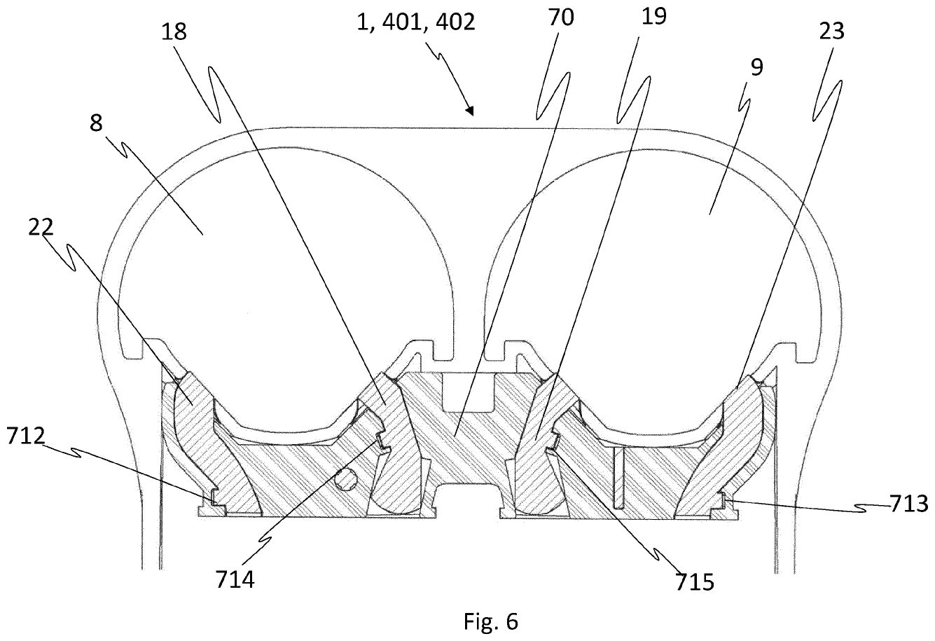

FIG. 6 shows an example of a positioning and supply device 1, 400, 402. In this example, the positioning and supply device 1, 400, 402 has a holder device 70 for holding the light guides, in particular two optical guide pairs.

Furthermore, in this example the holder device 70 has accommodation points 712, 714 for accommodating the first optical guide 18 and the second optical guide 22. Particularly preferably, the holder device 70 also has accommodation points 713, 715 for accommodating the further first optical guide 19 and the further second optical guide 23. The accommodation points 712, 714 (see FIG. 6) and/or 713, 715 are preferably an integral component of the first part 710 of the holder device 70 and/or the second part 711 of the holder device 70.

If the holder device 70 is designed to hold a plurality of optical guide pairs, the holder device 70 preferably has a radiation barrier 72 between the accommodation points for accommodating the first optical guide pair and the accommodation points for the second optical guide pair. The radiation barrier 72 is preferably an integral component of the holder device, in particular of the first part 710 of the holder device 70 and/or of the second part 711 of the holder device 70. The accommodation points 712, 714 and/or 713, 715 preferably hold the optical guides 18, 19, 22, 23 respectively in a positive fit and/or in a non-positive fit and/or in a material fit.

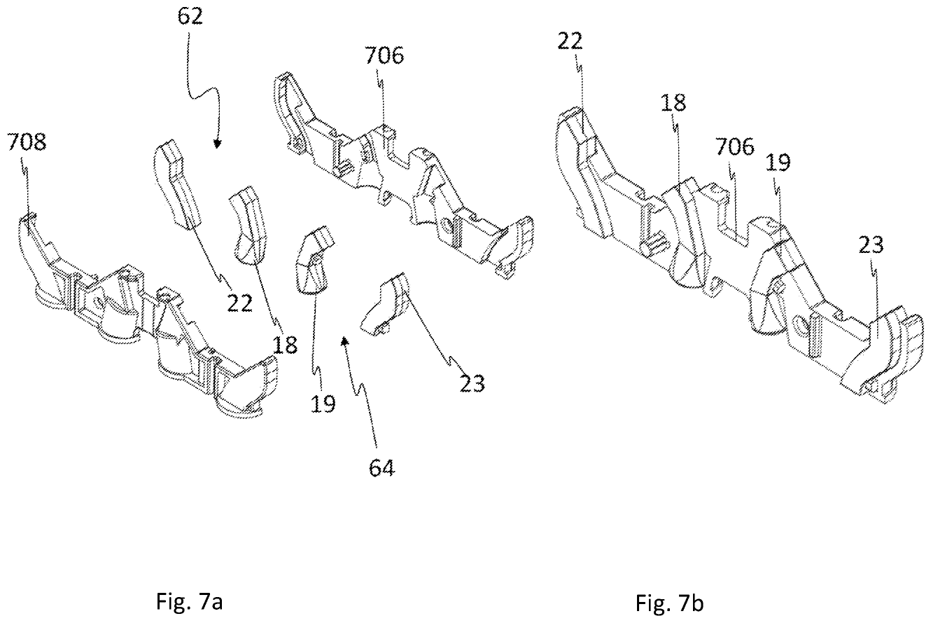

FIG. 7a shows an example of a first part 710 of the holder device 70 and/or a second part 711 of the holder device 70 as well as two optical guide pairs 62 and 64. In FIG. 7b, the two optical guide pairs 62 and 64 are shown as being in contact, in sections or only in section, with the first part 706 of the holder device 70.

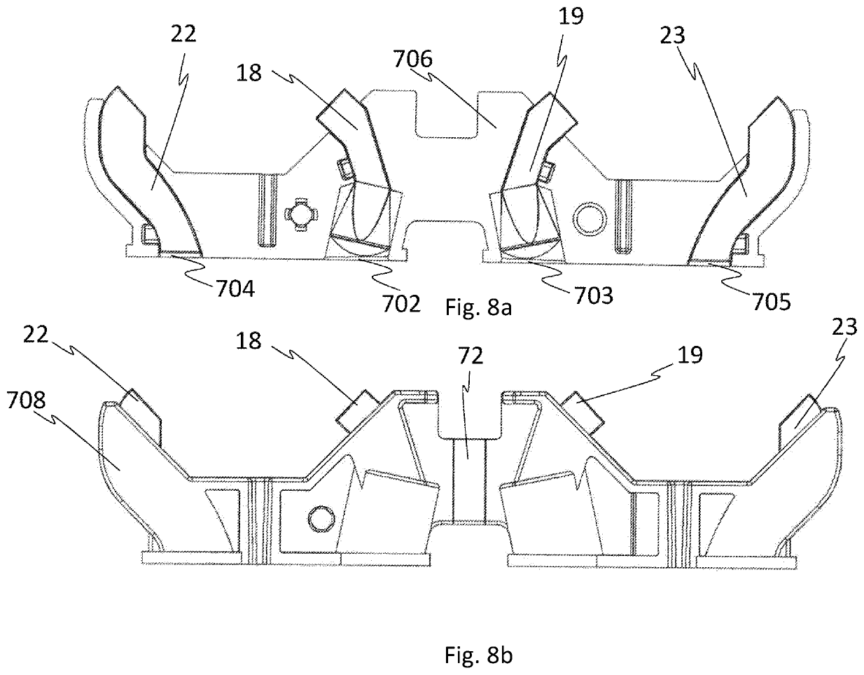

FIG. 8a shows a sectional view of a holder device 70. The holder device has two optical guide pairs 18, 22 and 19, 23. Furthermore, the holder device 70 has radiation incoupling points 702, 703 and radiation outcoupling points 704, 705. In this case, the radiation incoupling point 702 is embodied between the first optical guide 18 and the radiation source 76 in one usage configuration. The radiation outcoupling point 704 is arranged between the second optical guide 22 and the detection device 78 in one usage configuration. The holder device 70 can additionally have a second radiation incoupling point 703 and a second radiation outcoupling point 705. In one usage configuration, the second radiation incoupling point 703 is embodied between the further first optical guide 19 and the radiation source 76 or a further radiation source. In one usage configuration, the second radiation outcoupling point 705 is arranged between the further second optical guide 23 and the detection device 78 or a further detection device.

The holder device 70 preferably has a plurality of parts, one part can be, e.g., a rear holder part 706. Another part can be, e.g., a front holder part 708 (see FIG. 8b).

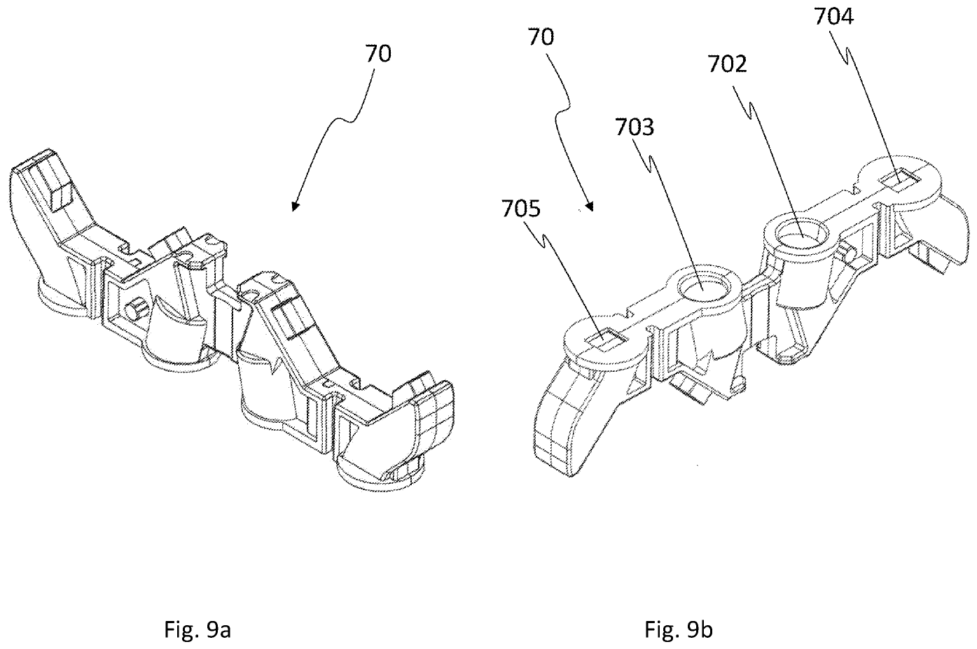

FIG. 9a shows a perspective elevation of an example of a holder device 70. The holder device 70 has two optical guide pairs.

FIG. 9b shows an underside of the holder device 70 shown in FIG. 9a. The holder device 70 can have a radiation incoupling point 702 and a radiation outcoupling point 704, and additionally or alternatively, the holder device can have a further first radiation incoupling point 703 and a further second radiation outcoupling point 705.

FIG. 10a shows an example of a positioning and supply device 1 and a holder device 70. The positioning and supply device 1 preferably has a holder device 709 for holding the holder device 70. The holder device 709 can be designed for holding the holder device 70 in a detachable manner or for holding it in a manner such that it cannot be detached without destroying it. The holder device 709 preferably has a first and/or a second part, which can interact with the holder device 70 in a positive fit, material fit, non-positive fit, and/or magnetic fit for holding the holder device 70.

FIG. 10b shows a state in which the holder device 70 is coupled to the positioning and exposure device 1, 400, 402, and in particular is arranged in a region that is partially or largely delimited by the guiding and support structure 6. The reference number 801 designates a coupling part for the positioning and exposure device 1, 400, 402, which is preferably embodied by the guiding and support structure 6, in particular in one piece. Reference numbers 82 and 83 identify schematically-represented force application devices, wherein at least one force application device is preferably provided for each examination area 8, 9.

FIG. 10c schematically shows an example of a supply device 74. This supply device 74 preferably has a further coupling part 802 as a component of the housing or of a housing part. The further coupling part 802 preferably detachably couples the first coupling part 801 of the positioning and exposure device 1, 400, 402.

The present invention thus relates to a positioning and exposure device 1 for the defined arrangement on at least one body part 2, 3 of a living being 4 and for exposing the body part 2, 3 to radiation for determining at least one vital parameter of the living being 4. The positioning and exposure device 1 preferably has at least:

A guiding and support structure 6 for delimiting an examination area 8, wherein the body part 2 can be positioned during exposure in the examination area 8, wherein the guiding and support structure 6 in a section 10 delimiting the examination area 8 forms at least one radiation entry area 12, wherein radiation can be introduced through the radiation entry area 12 into the examination area 8, and wherein the guiding and support structure 6 forms a radiation exit area 16 in a further section 14 delimiting the examination area 8, wherein at least part of the radiation which can be introduced through the radiation entry area 12 into the examination area 8 can be guided out of the examination area 8 through the radiation exit area 16. Preferably a first elongate optical guide 18 is arranged in the beam path 20 of the radiation at least upstream of the examination area 8, wherein the first optical guide 18 is curved at least in sections for at least one deflection of the beam path 20 of the radiation which can be introduced into the first optical guide 18. Additionally or alternatively, a second elongate optical guide 22 is arranged in beam path 20 of the radiation, at least downstream of radiation exit area 16, wherein the second optical guide 22 is curved at least in sections for at least one deflection of the beam path 20 of the radiation which can be introduced into second optical guide 22.

Additionally or alternatively, the first optical guide 18 has a first entry surface 24, a first main body 26, and a first exit surface 28, wherein the first main body 26 and the first exit surface 28 are oriented such that a main outward radiation direction 30 from the first optical guide 18 is defined and the second optical guide 22 has a second entry surface 32, a second main body 34, and a second exit surface 36, wherein the second main body 34 and the second entry surface 32 are oriented such that a main inward radiation direction 38 into the second optical guide 22 is defined, wherein main outward radiation direction 30 and the main inward radiation direction 38 are oriented relative to one another at an angle of between 65° and 115°, in particular at an angle of between 75° and 105° or at an angle of between 85° and 95°.

### LIST OF REFERENCE SYMBOLS

| | | | |
|---|---|---|---|
| 1 | Positioning and | 23 | Further second optical guide |
| | exposure device | 24 | First entry surface |
| 2 | Body part | 26 | First main body |
| 3 | Further body part | 28 | First exit surface |
| 4 | Living being | 30 | Main outward radiation direction |
| 6 | Guiding and support structure | 32 | Second entry surface |
| 8 | Examination area | 34 | Second main body |
| 9 | Further or second | 36 | Second exit surface |
| | examination area | 38 | Main inward radiation direction |
| 10 | Section delimiting the examination | 39 | Angle between main inward radiation |
| | area | | direction and main outward radiation |
| 12 | Radiation entry area | | direction |
| 13 | Further or second | 40 | First portion of the beam path |
| | radiation entry area | 42 | Second portion of the beam path |
| 14 | Further section adjacent to the | 44 | Deflection area |
| | examination area | 46 | Third portion of the beam path |
| 16 | Radiation exit area | 48 | Fourth portion of the beam path |
| 17 | Further or second | 50 | Second deflection region |
| | Radiation exit area | 54 | Tapering portion |
| 18 | First optical guide | 60 | Converging lens |
| 19 | Further first optical guide | 62 | First optical guide pair |
| 20 | Beam path | 64 | Second optical guide pair |
| 22 | Second optical guide | 183 | End of the conical or |
| 70 | Holder device | | tapering portion and beginning of the |
| 72 | Radiation barrier | | tubular or |
| 74 | Supply device | | cylindrical portion |
| 76 | Radiation source | 185 | Transition point |
| 78 | Detection device | 186 | Cylindrical and/or tubular |
| 80 | Coupling device | | and/or conical area |
| 82 | Force application device | 187 | Straight area |
| 83 | Further or second | 188 | Transition point |
| | force application device | 189 | Region |
| 84 | Measuring device | 210 | Lower bend on exterior contour |
| 86 | Carrier device | 211 | Upper bend on exterior contour |
| 88 | Control device | 212 | Lower straight accommodation |
| 89 | Accommodation space | | section on the interior contour |
| 90 | Functional fluid supply | 213 | End of the straight section 212 and |
| | device | | beginning of the |
| 92 | Supply line to the | | subsequent bent section 214 |
| | functional fluid supply | 214 | Curved section on the |
| | device | | interior contour |
| 94 | Supply line to the | 220 | Lower bend on exterior contour |
| | force application device | 221 | Straight portion with turning point to |
| 96 | Line connection | | exterior contour |
| 98 | Interface | 222 | Center bending portion on exterior |
| 99 | Cover glass | | contour |
| 100 | Further cover glass | 223 | Straight portion on exterior contour |
| 180 | Transition from lenticular | 224 | Upper bend on exterior contour |
| | portion on conical portion | 225 | Lower bend on interior contour |
| 181 | Flattened contour, in particular flat | 226 | Turning point on interior contour |
| | contour | 703 | Further or second |
| 182 | Tubular or cylindrical portion | | radiation incoupling point |
| 227 | Further bend towards interior contour | 704 | Radiation outcoupling point |
| | opposing lower bend | 705 | Further or second |
| 228 | Straight portion | | Radiation outcoupling point |
| 230 | Straight portion on exterior contour | 706 | Rear holder part |
| 231 | Curved portion on exterior contour | 708 | Front holder part |
| 232 | Straight portion on interior contour | 709 | Holder device for accommodating the |
| 233 | Bent portion on interior contour | | holder device |
| 301 | Angle a | 710 | First part of the holder device 709 |
| 302 | Distance b | 711 | Second part of the holder device |
| 303 | Height c | 709 | |
| 304 | Angle d | 712 | First accommodation point |
| 305 | Distance e | 713 | Further first accommodation point |
| 306 | Height f | 714 | Second accommodation point |
| 307 | Angle g | 715 | Further second accommodation point |
| 308 | Distance h | 801 | Coupling portion of the coupling point |
| 309 | Height i | | 80 for the |
| 400 | Second positioning and | | positioning and |
| | exposure device | | exposure device 1 |
| 402 | Third positioning and | 802 | Coupling portion of the |
| | exposure device | | Coupling point 80 for |
| 702 | Radiation incoupling point | | supply device 74 |

1. A positioning and exposure device (1) for the defined arrangement on at least one body part (2, 3) of a living being (4) and for exposing the body part (2, 3) to radiation for determining at least one vital parameter of the living being (4), comprising at least:
a guiding and support structure (6) for delimiting an examination area (8), wherein the body part (2) can be positioned during exposure in the examination area (8),
wherein the guiding and support structure (6) in a section (10) delimiting the examination area (8) forms at least one radiation entry area (12), wherein radiation can be introduced through the radiation entry area (12) into the examination area (8),
and
wherein the guiding and support structure (6) forms a radiation exit area (16) in a further section (14) delimiting the examination area (8), wherein at least part of the radiation which can be introduced through the radiation entry area (12) into the examination area (8) can be guided out of the examination area (8) through the radiation exit area (16),
and
wherein a first elongate optical guide (18) is arranged in the beam path (20) of the radiation at least upstream of the examination area (8), wherein the first optical guide (18) is curved at least in a section thereof for at least one deflection of the beam path (20) of the radiation which can be introduced into the first optical guide (18),
and/or
a second elongate optical guide (22) is arranged in the beam path (20) of the radiation, at least downstream of the radiation exit region (16), wherein the second optical guide (22) is curved at least in a section thereof for at least one deflection of the beam path (20) of the radiation which can be introduced into the second optical guide (22).

2. The positioning and exposure device (1) according to claim 1,
characterized in that
the first optical guide (18) comprises a first entry surface (24), a first main body (26), and a first exit surface (28), wherein the first main body (26) and the first exit surface (28) are oriented such that a main outward radiation direction (30) out of the first optical guide (18) is defined,
and
the second optical guide (22) comprises a second entry surface (32), a second main body (34), and a second exit surface (36), wherein the second main body (34) and the second entry surface (32) are oriented such that a main inward radiation direction (38) into the second optical guide (22) is defined,
wherein the main outward radiation direction (30) and the main inward radiation direction (38) are oriented to one another at an angle of between 65° and 115°, in particular at an angle of between 75° and 105° or at an angle of between 85° and 95°.

3. A positioning and exposure device (1) for the defined arrangement on at least one body part (2, 3) of a living being (4) and for exposing the body part (2, 3) to radiation for determining at least one vital parameter of the living being (4),
comprising at least:
a guiding and support structure (6) for delimiting an examination area (8), wherein the body part (2, 3) can be positioned during exposure in the examination area (8),
wherein the guiding and support structure (6) in a section (10) delimiting the examination area (8) forms at least one radiation entry area (12), wherein radiation can be introduced through the radiation entry area (12) into the examination area (8),
and
wherein the guiding and support structure (6) forms a radiation exit area (16) in a further section (14) delimiting the examination area (8), wherein radiation that can be introduced into the examination area (8) through the radiation entry area (12) can be guided out of the examination area (8) through the radiation exit area (16),
the first optical guide (18) comprises a first entry surface (24), a first main body (26), and a first exit surface (28), wherein the first main body (26) and the first exit surface (28) are oriented such that a main outward radiation direction (30) out of the first optical guide (18) is defined,
and
wherein the second optical guide (22) comprises a second entry surface (32), a second main body (34), and a second exit surface (36), wherein the second main body (34) and the second entry surface (32) are oriented in such a way that a main inward radiation direction (38) into the second optical guide (22) is defined,
wherein the main outward radiation direction (30) and the main inward radiation direction (38) are oriented to one another at an angle of between 65° and 115°, in particular at an angle of between 75° and 105° or at an angle of between 85° and 95°.

4. The positioning and exposure device (1) according to claim 3, characterized in that a first elongate optical guide (18) is arranged in the beam path (20) of the radiation at least upstream of the examination area (8), wherein the first optical guide (18) is curved at least in a section thereof for at least one deflection of the beam path (20) of the radiation which can be introduced into the first optical guide (18),
and/or
a second elongate optical guide (22) is arranged in the beam path (20) of the radiation at least downstream of the radiation exit region (16), wherein the second optical guide (22) is curved at least in a section thereof for at least one deflection of the beam path (20) of the radiation which can be introduced into the second optical guide (22).

5. The positioning and exposure device (1) according to one of the preceding claims, characterized in that
at least the first optical guide (18) and/or the second optical guide (229) is/are fiber-free,
and
wherein due to the curvature of the first optical guide (18), a first portion (40) of the beam path (20) is oriented relative to a second portion (42) of the beam path (20) at an angle of more than 5°, in particular of more than 10° or more than 15°, relative to a second portion (42) of the beam path (20), in particular in a range between 5° and 85° and preferably in a range between 20° and 75° or in a range between 30° and 60°,
wherein the first portion (40) of the beam path (20) extends in the course of the beam path (20) at least immediately upstream of a first deflection region (44), and the second portion (42) of the beam path (20) in the course of the beam path (20) extends at least immediately downstream of the first deflection region (44),
wherein the deflection region (44) is designed as a component of the first optical guide (18), in particular as a surface or coating,
and/or
wherein due to the curvature of the second optical guide (22), a third portion (46) of the beam path (20) is oriented relative to a fourth portion (48) of the beam path (20) at an angle of more than 5°, in particular of more than 10° or more than 15°, in particular in a range between 5° and 85° and preferably in a range between 20° and 75° or in a range between 30° and 60°, wherein with the third portion (46) of the beam path (20) extends in the course of the beam path (20) at least immediately upstream a second deflection region (50), and wherein the fourth portion (48) of the beam path (20) extends in the course of the beam path (20) at least immediately downstream of the second deflection region (22),
wherein the second deflection region (50) is designed as a component of the second optical guide (22), in particular as a surface or coating.

6. The positioning and exposure device (1) according to one of the preceding claims, characterized in that
the first optical guide (18) has portions with differently sized cross-sectional areas in its longitudinal direction, wherein the cross-sectional area in the region of the first entry surface (24) is larger than in a central portion formed between the first entry surface (24) and the first exit surface (28), the first optical guide (18) preferably forms a tapering portion (54) extending from the first entry surface (24) in the direction of the first exit surface (28), wherein the sectional surface decreases, in particular continuously, starting from the first
entry surface (24), in the direction of the first exit surface (28).

7. The positioning and exposure device (1) according to one of the preceding claims, characterized in that
at least a first surface portion (44) of the first optical guide (18) and/or at least a second surface portion (50) of the second optical guide (22) is matted, structured and/or coated.

8. The positioning and exposure device (1) according to one of the preceding claims, characterized in that
the first optical guide (18) and the second optical guide (22) differ in at least one of the following parameters: Shape, in particular curvature, volume, mass, length, and/or material,
and/or
the optical guide (18) has a converging lens (60), wherein the converging lens (60) forms the first entry surface (24),
and/or
two optical guide pairs (62, 64) are provided, wherein the first optical guide pair (62) is formed by the first optical guide (18) and the second optical guide (22) and the second optical guide pair (64) is formed by a further first optical guide (19) and the second optical guide (22) or a further second optical guide (23), wherein the first optical guide pair (62) and the second optical guide pair (64) are arranged such that different body parts (2, 3), in particular fingers, are alternately exposed to radiation.

9. The positioning and exposure device (1) according to one of the preceding claims, characterized in that
a holder device (70) is provided, wherein the holder device (70) is designed for the defined arrangement of at least two optical guide pairs (62, 64),
wherein the holder device (70) comprises a functional material, in particular pigment, wherein the functional material absorbs radiation in the wavelength range of at least 500 nm to 1040 nm,
and/or
wherein the holder device (70) encloses the optical guides (18, 22, 19, 23), at least in a section thereof, on at least two sides, at least in sections, preferably largely, in particular more than 50% (based on the surface area of the specific optical guide) or more than 75% or more than 90% or completely,
and/or
the second optical guide (22) and the further second optical guide (23) are spaced closer to one another than the second optical guide (22) is spaced from the further first optical guide (19) or the first optical guide (18) is spaced from the further second optical guide (23),
and/or
the optical guides (18, 22, 19,23) are arranged in the holder device (70) such that the axial center of the individual optical guides (18, 22, 19,23) extend in the same plane or in planes which are spaced apart by less than 20 mm, in particular less than 10 mm or less than 5 mm or less than 1 mm,
and/or
the holder device (70) comprises a radiation barrier (72), in particular a wall, between the second light device (22) and the further second light device (22), wherein the radiation barrier (72) prevents the further second optical guide (23) from being acted upon by leakage radiation which escapes from the first optical guide (18) or prevents the second optical guide (22) from being acted upon by leakage radiation which escapes from the further first optical guide (19).

10. The positioning and exposure device (1) according to one of the preceding claims, characterized in that
at least a first radiation source (76) and a first detection device (78), in particular a radiation detection device, are arranged in a supply device (74),
wherein the radiation source (76) is arranged upstream of the first entry surface (24) in the beam path (20) of the radiation that can be generated by the first radiation source (76),
and
wherein the detection device (78) is arranged downstream of the second exit surface (36) in the beam path (20) of the radiation that can be generated by the first radiation source (76).

11. The positioning and exposure device (1) according to claim 9, characterized in that a coupling device (80) is provided for detachably coupling the guiding and support structure (6) and the supply device (74) at least in a form fit and/or at least in a friction contact,
wherein at least the first optical guide (18) is arranged on the guiding and support structure (6) in a decoupled state.

12. The positioning and exposure device (1) according to claim 9, characterized in that a coupling device (80) is provided for detachably coupling the guiding and support structure (6) and the supply device (74) at least in a form fit and/or at least in a friction contact, wherein at least the first optical guide (18) is arranged at the supply device (74) in a decoupled state.

13. The positioning and exposure device (1) according to one of the preceding claims, characterized in that
a force application device (82) is provided for exposing the body part to pressure, wherein the force application device (82) is connected to and supported by the guiding and support structure (6).

14. A supply device (74) for providing radiation, in particular for determining the vital parameters of a living being, wherein the supply device (74) comprises at least:
A first radiation source (76) and a first detection device (78), in particular a radiation detection device,
a first optical guide and a second optical guide,
wherein the first optical guide (18) is arranged such that the radiation from the radiation source (76) can be introduced into the first optical guide (18) through a first entry surface (24) of the first optical guide (18), wherein the radiation can be guided out of the first optical guide (18) through a first exit surface (28) of the first optical guide (18), wherein the second optical guide (22) is arranged such that radiation that has exited the first optical guide (18) through the first exit surface (28) of the first optical guide (18) can be introduced into the second optical guide (22) through a second entry surface (32) of the second optical guide (22), wherein the radiation introduced into the second optical guide (22) through the second entry surface (32) can be guided out of the second optical guide (22) through a second exit surface (36) and can be conducted to the detection device (78),
wherein the first optical guide (18) and/or the second optical guide (22) is/are curved, and/or
wherein a first main body (26) extends between the first entry surface (24) and the first exit surface (28), wherein the first main body (26) and the first exit surface (28) are oriented such that a main outward radiation direction (30) is defined, and wherein a second main body (34) extends between the second entry surface (32) and the second exit surface (36), wherein the second main body (34) and the second exit surface (36) are oriented such that a main inward radiation direction (38) is defined, wherein the main outward radiation direction (30) and the inward direction of radiation (38) are oriented to one another at an angle of between 65° and 115°, in particular at an angle of between 75° and 105° or at an angle of between 85° and 95°.

15. A measuring device (84) for determining at least one vital parameter of a living being (4), in particular for continuously determining the intra-arterial blood pressure on at least one finger (2, 3) of a hand, comprising at least:
a supply device (74) for supplying at least one radiation source (76) for providing radiation and for providing at least one detection device (78),
and
a positioning and exposure device (1) for the defined arrangement on at least one body part (2, 3) of a living being (4) and for exposing the body part (2, 3) to the radiation, wherein the positioning and exposure device (1) comprises a guiding and support structure (6) for delimiting an examination area (8),
wherein the supply device (74) and the positioning and exposure device (1) can be physically coupled to and/or decoupled from one another, preferably without the use of tools and/or non-destructively,
and a first optical guide and a second optical guide,
wherein the first optical guide (18) is arranged such that the radiation from the radiation source (76) can be introduced into the first optical guide (18) through a first entry surface (24) of the first optical guide (18), wherein the radiation can be guided out of the first optical guide (18) through a first exit surface (28) of the first optical guide (18),
wherein the second optical guide (22) is arranged such that radiation that has exited the first optical guide (18) through the first exit surface (28) of the first optical guide (18) can be introduced into the second optical guide (22) through a second entry surface (32) of the second optical guide (22), wherein the radiation introduced into the second optical guide (22) through the second entry surface (32) can be guided out of the second optical guide (22) through a second exit surface (36) and can be conducted to the detection device (78),
wherein the first optical guide (18) and/or the second optical guide (22) is/are curved, and/or
wherein a first main body (26) extends between the first entry surface (24) and the first exit surface (28), wherein the first main body (26) and the first exit surface (28) are oriented such that a main outward radiation direction (30) is defined, and wherein a second main body (34) extends between the second entry surface (32) and the second exit surface (36), wherein the second main body (34) and the second exit surface (36) are oriented such that a main inward radiation direction (38) is defined, wherein the main outward radiation direction (30) and the inward direction of radiation (38) are oriented to one another at an angle of between 65° and 115°, in particular at an angle of between 75° and 105° or at an angle of between 85° and 95°.

16. A method for exposing living beings (4) to radiation, in particular for determining at least one vital parameter, comprising at least the steps of:
providing a measuring device (84) for determining at least one vital parameter of a living being (4), in particular for continuously determining the intra-arterial blood pressure on at least one finger (2, 3) of a hand, comprising at least one supply device (74) for providing at least one radiation source (76) for supplying radiation and for providing at least one detection device (78),
and
a positioning and exposure device (1) for the defined arrangement on at least one body part (2, 3) of a living being (4) and for exposing the body part (2, 3) to the radiation, wherein the positioning and exposure device (1) comprises a guiding and support structure (6) for delimiting an examination area (8),
wherein the supply device (74) and the positioning and exposure device (1) can be physically coupled to and/or decoupled from one another, preferably without the use of tools and/or non-destructively,
and
a first optical guide (18) and a second optical guide (22),
wherein the first optical guide (18) is arranged such that the radiation from the radiation source (76) can be introduced into the first optical guide (18) through a first entry surface (24) of the first optical guide (18), wherein the radiation can be guided out of the first optical guide (18) through a first exit surface (28) of the first optical guide (18), wherein the second optical guide (22) is arranged such that at least part of the radiation that has exited the first optical guide (18) through the first exit surface (28) of the first optical guide (18) can be introduced into the second optical guide (22) through a second entry surface (32) of the second optical guide (22), wherein the radiation introduced into the second optical guide (22) through the second entry surface (32) can be guided out of the second optical guide (22) through a second exit surface (36) and can be conducted to the detection device (78),
wherein the first optical guide (18) and/or the second optical guide (22) is/are curved,
and/or
wherein a first main body (26) extends between the first entry surface (24) and the first exit surface (28), wherein the first main body (26) and the first exit surface (28) are oriented such that a main outward radiation direction (30) is defined, and wherein a second main body (34) extends between the second entry surface (32) and the second exit surface (36), wherein the second main body (34) and the second exit surface (36) are oriented such that a main inward radiation direction (38) is defined,
wherein the main outward radiation direction (30) and the main inward radiation direction (38) are oriented to one another at an angle of between 65° and 115°, in particular at an angle of between 75° and 105° or at an angle of between 85° and 95°, emitting radiation by the radiation source (76),
directing the radiation to an examination area (8), wherein the radiation is guided through the first optical guide (18),
introducing radiation from the examination area (8) into the second optical guide (22), conducting the radiation introduced into the second optical guide (22) to the detection device (78).

17. A method for exposing living beings (4) to radiation, in particular for determining at least one vital parameter, comprising at least the steps of:
providing a positioning and exposure device (1),
wherein the positioning and exposure device (1) has a guiding and support structure (6) for delimiting an examination area (8), wherein the body part (2, 3) is positioned in the examination area (8),
wherein the guiding and support structure (6) forms at least one radiation entry area (12) in a section (10) delimiting the examination area (8),
wherein the guiding and support structure (6) forms a radiation exit area (16) in a further section (14) delimiting the examination area (8),
and
wherein a first elongate optical guide (18) is arranged in the beam path (20) of the radiation at least upstream of the examination area (8), wherein the first optical guide (18) is curved at least in sections for at least one deflection of the beam path (20) of the radiation which can be introduced into the first optical guide (18),
and/or
wherein a second elongate optical guide (22) is arranged in the beam path (20) of the radiation, at least downstream of the radiation exit region (16), wherein the second optical guide (22) is curved at least in sections for at least one deflection of the beam path (20) of the radiation which can be introduced into the optical guide (22),
Arranging in a defined manner at least one body part (2, 3) of a living being (4) on or in the positioning and exposure device (1), and,
exposing the body part (2, 3) to radiation,
wherein radiation is introduced through the first radiation entry area (12) and out of the first optical guide (18) into the examination area (8) for exposing the body part (23), and,
wherein at least part of the radiation introduced into the examination area (8) through the radiation entry area (12) is conducted through the radiation exit area (16) and into the second optical guide (22), wherein the radiation introduced into the second optical guide (22) is conducted from the second optical guide (22) in a defined manner.

18. A method for exposing living beings (4) to radiation, in particular for determining at least one vital parameter, comprising at least the steps of:
providing a positioning and exposure device (1),
wherein the positioning and exposure device (1) has a guiding and support structure (6) for delimiting an examination area (8), wherein the body part (2, 3) is positioned in the examination area (8),
wherein the guiding and support structure (6) forms at least one radiation entry area (12) in a section (10) delimiting the examination area (8),
wherein the guiding and support structure (6) forms a radiation exit area (16) in a further section (14) delimiting the examination area (8),
and,
wherein a first elongate optical guide (18) is arranged in the beam path (20) of the radiation at least upstream of the examination area (8), wherein the first optical guide (18) has a first entry surface (24), a first main body (26), and a first exit surface (28), wherein the first main body (26) and the first exit surface (28) are oriented such that a main outward radiation direction (30) is defined,
and,
wherein a second elongate optical guide (22) is arranged in the beam path (22) of the radiation at least downstream of the radiation exit region (16), wherein the second optical guide (22) has a second entry surface (32), a second main body (34), and a second exit surface (36), wherein the second main body (34) and the second entry surface (32) are oriented such that a main inward radiation direction (38) is defined, wherein the main outward radiation direction and the main inward radiation direction are oriented relative to one another (at an angle of between 65° and 115°, in particular at an angle of between 75° and 105° or at an angle of between 85° and 95°,
Arranging in a defined manner at least one body part (2, 3) of a living being (4) on the positioning and exposure device (1), and,
Exposing the body part (2, 3) to radiation,
wherein radiation is introduced through the first radiation entry area (12) and out of the first optical guide (18) into the examination area (8) for exposing the body part (2, 3),
and,
wherein at least part of the radiation introduced into the examination area (8) through the radiation entry area (12) is conducted through the radiation exit area (16) and into the second optical guide (22), wherein the radiation introduced into the second optical guide (22) is conducted from the second optical guide (22) in a defined manner.

19. A method for providing radiation, in particular for determining at least one vital parameter, at least comprising the steps of:
providing a supply device (74) for supplying radiation and a functional fluid, wherein the supply device has at least:
a radiation source (76) and a detection device (78), in particular a radiation detection device,
a first optical guide (18) and a second optical guide (22),
wherein the first optical guide (18) is arranged such that the radiation from the radiation source (76) can be introduced into the first optical guide (18) through a first entry surface (24) of the first optical guide (18), wherein the radiation can be guided out of the first optical guide (18) through a first exit surface (28) of the first optical guide (18),
wherein the second optical guide (22) is arranged such that radiation that has exited the first optical guide (18) through the first exit surface (28) of the first optical guide (18) can be introduced into the second optical guide (22) through a second entry surface (32) of the second optical guide (22), wherein the radiation introduced into the second optical guide (22) through the second entry surface (32) can be guided out of the second optical guide (22) through a second exit surface (36) and can be conducted to the detection device (78),
wherein the first optical guide (18) and/or the second optical guide (22) is/are curved,
and/or
wherein a first main body (26) extends between the first entry surface (24) and the first exit surface (28), wherein the first main body (26) and the first exit surface (28) are oriented such that a main outward radiation direction (30) is defined, and wherein a second main body (34) extends between the second entry surface (32) and the second exit surface (36), wherein the second main body (34) and the second exit surface (36) are oriented such that a main inward radiation direction (38) is defined, wherein the main outward radiation direction (30) and the inward direction of radiation (38) are oriented to one another at an angle of between 65° and 115°, in particular at an angle of between 75° and 105° or at an angle of between 85° and 95°.
emitting radiation by the radiation source (76) and coupling of the radiation into the first optical guide (18) and
and,
detecting radiation by means of the detection device (78), wherein portions of the radiation emitted by the radiation source (76) and coupled into the first optical guide (18) are conducted to the detection device (78) through the second optical guide (22).

20. A set,
comprising at least a first positioning and exposure device according to any one of claims 1 to 9 and at least a second positioning and exposure device according one of claims 1 to 9, wherein the shape and/or orientations of the optical guides of the first positioning and exposure device is different from the shape and/or orientations of the optical guides of the second positioning and exposure device.
